(19) United States
(12) Patent Application Publication
(10) Pub. No.: US 2018/0238881 A1
(43) Pub. Date: Aug. 23, 2018

(54) RAPID IMMUNOCHROMATOGRAPHIC LATERAL FLOW ASSAY FOR EARLY ZIKA DISEASE DETECTION

(71) Applicant: Lusys Laboratories, Inc., San Diego, CA (US)

(72) Inventors: Weizhao LU, San Diego, CA (US); James Jingyu LU, San Diego, CA (US)

(21) Appl. No.: 15/895,861

(22) Filed: Feb. 13, 2018

Related U.S. Application Data
(60) Provisional application No. 62/600,089, filed on Feb. 14, 2017.

(51) Int. Cl. G01N 33/569; G01N 33/543

(52) U.S. Cl. CPC G01N 33/56983; G01N 2333/185; G01N 2800/26; G01N 33/54386

(57) ABSTRACT

The invention relates to a sensitive and specific rapid immunochromatographic lateral flow assay for Zika virus infection. The rapid assay of the invention can determine early, intermediate, and late Zika virus infection status.

### CROSS-REFERENCE

This application claims the benefit of U.S. Provisional Application No. 62/600,089, filed Feb. 14, 2017, which application is incorporated herein by reference.

### BACKGROUND OF THE INVENTION

The Zika virus, a flavivirus related to the dengue, West Nile, and yellow fever viruses, causes Zika virus disease in humans. Zika virus disease usually presents with mild symptoms lasting from several days to a week that can include one or more of fever, rash, joint pain, and conjunctivitis, but its symptoms can go unnoticed by the patient. The Zika virus is transmitted to humans by Aedes species mosquitoes, and can be transmitted from human to human by saliva, sexual intercourse, and from mother to baby in utero. During the last decade the Zika virus has spread from Africa and Asia to the Americas.

Zika virus disease requiring hospitalization is uncommon, but infection of a fetus can have devastating effects. Congenital Zika Syndrome in babies is characterized by features including microcephaly and severe abnormalities in neurological development. Currently no vaccines or drugs are available to prevent or treat infection.

Zika disease symptoms are often mistaken for those of other diseases, including viral infections that do not have the same effects on the developing fetus. Unfortunately, existing Zika virus diagnostic methods are commonly cross-reactive with other flavivirus infections due to the genetic similarity among flaviridae family members. For example, more than 50% of the genomic sequences of the Zika and dengue viruses are the same. Existing PCR and ELISA tests are not free of cross-reactivity with other viruses, and additionally require sophisticated equipment and experienced operators to carry out properly. PCR techniques also are limited by declining Zika RNA levels as infection progresses. A high percentage of Zika infections occur in developing countries where advanced diagnostic facilities and medical personnel are scarce or not available. In these regions, a diagnostic tool for determining Zika infection status that is inexpensive and easy to use is particularly needed.

### SUMMARY OF THE INVENTION

The present invention relates to compositions and methods for immunochromatographic analysis of a patient samples using a combination lateral flow test-strip assay to accurately and rapidly determine early, intermediate, or late Zika virus infection status in the patient. The methods of the invention combine a highly specific Zika antibody sandwich assay with an anti-Zika antibody subtype assay. The combination format of these two assays as described herein provides a sophisticated Zika virus infection diagnostic tool in a rapid test. The compositions of the invention provide the assay, a device comprising the assay, and kits comprising the device.

In particular, the present inventive compositions and methods are useful for determining infection status based on anti-Zika antibody subtype profile. The assay methods and compositions invention allows very specific determination of early, intermediate, and late Zika virus infection status. The compositions and methods of the invention thus allow determination of Zika virus infection status with very low cross-reactivity to related viruses.

The methods and compositions of the invention are economical and easy to use, for example by an individual in a home setting using a small body fluid sample obtained without professional assistance. Results are available in only 5 to 20 minutes, and are useful to patients who wish to avoid infecting others, as well as to practitioners and epidemiologists seeking to limit disease spread and end the Zika disease epidemic.

Specifically, the present invention provides a method for rapid assay of a human patient sample to determine Zika virus infection status in the patient, comprising: comparing a test signal produced at a first test line on a first lateral flow test strip exposed to the patient sample, and a test signal produced at a second test line on a second lateral flow test strip exposed to the patient sample, wherein: a) the presence of the test signal produced at the first test line indicates the presence of an anti-Zika antibody in the patient sample; and b) the presence of the test signal produced at the second test line indicates the presence of one of an anti-Zika-IgG or an anti-Zika IgM in the patient sample; wherein the test signal at each of the first and second test lines is produced in five to twenty minutes following application of the patient sample to a sample loading pad on the corresponding lateral flow test strip, and wherein when the test signal produced at the second test line indicates the presence of anti-Zika IgG in the patient sample, the Zika virus infection status determined is selected from: no Zika virus infection and early Zika virus infection, and wherein when the test signal produced at the second test line indicates the presence of anti-Zika IgM in the patient sample, the Zika virus infection status determined is selected from: no Zika virus infection and late Zika virus infection.

In embodiments, the test signal at the first test line that indicates the presence of the anti-Zika antibody in the patient sample is produced by binding of the anti-Zika antibody to a first Zika tracer antigen and to a first immobilized Zika detection antigen on the first test line, whereby a complex comprising the first Zika tracer antigen—anti-Zika antibody—first immobilized Zika detection antigen is formed and produces a test signal at the first test line.

In embodiments of the above method the test signal produced at the second test line:

i. indicates the presence in the patient sample of an anti-Zika IgG, wherein the test signal at the second test line is produced by binding of the anti-Zika IgG to a second Zika tracer antigen on the second lateral flow test strip and to an immobilized anti-human-IgG antibody on the second test line, whereby a complex comprising the second Zika tracer antigen—anti-Zika IgG—immobilized anti-human-IgG antibody is formed, thereby producing a test signal at the second test line; or

ii. indicates the presence in the patient sample of an anti-Zika IgM, wherein the test signal at the second test line is produced by binding of the anti-Zika IgM to a second Zika tracer antigen on the second lateral flow test strip and to an immobilized anti-human-IgM antibody on the second test line, whereby a complex comprising the second Zika tracer antigen—anti-Zika IgM—immobilized anti-human-IgM antibody is formed, thereby producing a test signal at the second test line.

In embodiments of (i) above, the second lateral flow test strip further comprises a third test line that is separate from the second test line, wherein the presence of a test signal produced at the third test line indicates the presence of an anti-Zika IgM in the patient sample, wherein the test signal at the third test line is produced by binding of the anti-Zika IgM to the second Zika tracer antigen on the second lateral flow test strip and to an immobilized anti-human-IgM antibody on the third test line, whereby a complex comprising the second Zika tracer antigen—anti-Zika IgM—immobilized anti-human-IgM antibody is formed thereby producing a test signal at the third test line, and wherein the Zika virus infection status determined is selected from: no Zika virus infection, early Zika virus infection, intermediate Zika virus infection, and late Zika virus infection. In related embodiments, determination of early Zika virus infection is made based on the presence of an anti-Zika antibody test signal at the first test line, the absence of an anti-Zika IgG test signal at the second test line, and the presence of an anti-Zika IgM test signal at the third test line. In related embodiments, determination of intermediate Zika virus infection is made based on the presence of an anti-Zika antibody test signal at the first test line, the presence of an anti-Zika IgG test signal at the second test line, and the presence of an anti-Zika IgM test signal at the third test line. In related embodiments, determination of late Zika virus infection is made based on the presence of an anti-Zika antibody test signal at the first test line, the presence of an anti-Zika IgG test signal at the second test line, and the absence of an anti-Zika IgM test signal at the third test line.

In related embodiments of any of these methods, the first tracer antigen is Zika NS1, the first immobilized Zika detection antigen on the first test line is Zika NS1, and the second tracer antigen is Zika NS1. In embodiments, the NS1 is recombinantly produced.

In embodiments, the first Zika tracer antigen and the first immobilized Zika detection antigen on the first test strip are the same or different. In embodiments, the second Zika tracer antigen on the second test strip is the same as or different from either of the first Zika tracer antigen and the first immobilized Zika detection antigen.

In embodiments of (i) above, determination of early Zika virus infection is made based on the presence of an anti-Zika antibody test signal at the first test line and the absence of an anti-Zika IgG test signal at the second test line.

In embodiments of (ii) above, determination of late Zika virus infection is made based on the presence of an anti-Zika antibody test signal at the first test line and the absence of an anti-Zika IgM test signal at the second test line.

In embodiments, the determination of no Zika virus infection is based on the absence of an antibody test signal at the first test line. In embodiments, the determination of early Zika virus infection, intermediate Zika virus infection, or late Zika virus infection does not represent infection with Dengue, Chikungunya, yellow fever, malaria, or West Nile virus.

The invention further provides an assay for determining Zika virus infection status of a patient by analyzing a sample obtained from the patient, comprising: (a) a first lateral flow test strip comprising, in the direction of flow, a sample application pad, a conjugate pad comprising a first Zika tracer antigen, a first test line comprising a first immobilized Zika detection antigen, and a first control line, wherein in the presence of an anti-Zika antibody in the patient sample a complex comprising the first Zika tracer antigen—anti-Zika antibody—first immobilized Zika detection antigen is formed and produces a test signal at the first test line; and (b) a second lateral flow test strip comprising, in the direction of flow, a sample application pad, a conjugate pad comprising a second Zika tracer antigen, a second test line comprising an immobilized anti-human-antibody specific for one of a human IgG and a human IgM, and a second control line, wherein in the presence of an anti-Zika IgG or IgM, respectively, in the patient sample, a complex comprising the second Zika tracer antigen—anti-Zika antibody—immobilized antibody specific for the human IgG or IgM is formed and produces a test signal at the second test line; wherein the first and second Zika tracer antigens are the same or different, and wherein when the immobilized anti-human antibody is specific for human IgG the Zika virus infection status determined is selected from: no Zika virus infection and early Zika virus infection, and wherein when the immobilized anti-human antibody is specific for human IgM the Zika virus infection status determined is selected from: no Zika virus infection and late Zika virus infection.

In embodiments of this assay, the second lateral flow test strip further comprises, beyond the conjugate pad in the direction of sample flow, and separate from the second test line, a third test line comprising an immobilized anti-human-IgM antibody, wherein in the presence of an anti-Zika IgM in the patient sample a complex comprising the second Zika tracer antigen—anti-Zika IgM—immobilized anti-human-IgM antibody is formed and produces a test signal at the third test line, wherein the third Zika tracer antigen is the same or different from the first and second Zika tracer antigens, and wherein the Zika virus infection status determined is selected from: no Zika virus infection, early Zika virus infection, intermediate Zika virus infection, and late Zika virus infection.

In embodiments of the provided assay, the first and second lateral flow test strips are present together in a single housing. In embodiments, the assay further comprises a vessel containing an amount of chase buffer sufficient to properly operate the assay when the chase buffer is loaded on the sample pad of each of the first and second test strips after loading the patient sample.

In embodiments of any of the above methods or assays, the patient sample is whole blood, serum, plasma, saliva, semen, urine, or vaginal fluids. In embodiments, the patient sample is 5 uL to 40 uL whole blood, 2.5 uL to 20 uL serum or plasma, or 5 uL to 40 uL saliva.

The invention also provides a method for rapid assay of a human patient sample to determine Zika virus infection status in the patient, comprising: comparing a test signal produced at a first test line on a first lateral flow test strip exposed to the patient sample, a test signal produced at a second test line on a second lateral flow test strip exposed to the patient sample, and a test signal produced at a third test line on the second lateral flow test strip exposed to the patient sample, wherein: (a) the presence of the test signal produced at the first test line indicates the presence of an anti-Zika antibody in the patient sample; (b) the presence of the test signal produced at the second test line indicates the presence of an anti-Zika-IgG in the patient sample; and (c) the presence of the test signal produced at the third test line indicates the presence of an anti-Zika IgM in the patient sample; wherein the test signal at each of the first, second, and third test lines is produced in five to twenty minutes following application of the patient sample to a sample loading pad on the corresponding lateral flow test strip, and wherein the Zika virus infection status determined is selected from: no Zika virus infection, early Zika virus infection, intermediate Zika virus infection, and late Zika virus infection.

In embodiments of the assays described above, the first lateral flow test strip comprises a first control line, wherein the first control line is separate from the first test line, the second lateral flow test strip comprises a second control line that is separate from the second test line, or both. In embodiments, the first control line comprises a first control line immobilized detection antigen, and the second control line comprises a second control line immobilized detection antigen. In specific embodiments, the first tracer antigen is the Zika NS1 protein, and the first control line immobilized detection antigen is anti-Zika NS1 antibody. In certain embodiments, the second tracer antigen is the Zika NS1 protein, and the second control line immobilized detection antigen is anti-Zika NS1 antibody. In embodiments, the Zika NS1 protein is recombinantly produced. In embodiments, the anti-Zika NS1 antibody is a monoclonal antibody.

The invention further provides a device comprising an assay described above, and a test kit comprising such a device.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings.

FIGS. 1A to 1C. Zika Lateral Flow Assay Test Strip Example Detail. FIG. 1A. First Lateral Flow Test Strip (Anti-Zika Antibody Sandwich Assay Test Strip). The lateral flow test strip zones indicated comprise as follows: Zone 1—sample loading pad; Zone 2—conjugate pad comprising a first Zika tracer reagent; Zone 3—first test line comprising a first immobilized Zika detection reagent; Zone 4—First Control Line; Zone 5—absorbent pad. FIG. 1B. Second Lateral Flow Test Strip (Anti-Zika IgG or IgM Subtype Assay). The lateral flow test strip zones indicated comprise as follows: Zone 1—sample loading pad; Zone 2—conjugate pad comprising a second Zika tracer reagent; Zone 3—second test line comprising an immobilized anti-human-IgG or IgM antibody; Zone 4—Second Control Line; Zone 5—absorbent pad. FIG. 1C. Second Lateral Flow Test Strip (Anti-Zika IgG and IgM Subtype Assay). The lateral flow test strip zones indicated comprise as follows: Zone 1—sample loading pad; Zone 2—conjugate pad comprising a second Zika tracer reagent; Zone 3A—second test line comprising immobilized anti-human-IgG antibody; Zone 3B—third test line comprising immobilized anti-human-IgM antibody; Zone 4—Second Control Line; Zone 5—absorbent pad.

FIGS. 2A and 2B. Determination of Early Zika Virus Infection Status—Positive Test Pattern Examples. FIG. 2A. FIG. 2A shows a test pattern indicating determination of early Zika infection status (anti-Zika antibody positive, anti-Zika IgG negative), in a patient sample using an assay cassette comprising a first lateral flow test strip (as in FIG. 1A) and a second lateral flow test strip (IgG strip as in FIG. 1B). FIG. 2B. FIG. 2B shows a positive test pattern indicating determination of early Zika infection (anti-Zika antibody positive, anti-Zika IgM positive, IgG negative), in a patient sample using an assay cassette comprising a first lateral flow test strip and a second lateral flow test strip (IgG and IgM strip as in FIG. 1C).

FIGS. 3A to 3C. Determination of Zika Virus Infection Status—Negative Test Pattern Examples. FIG. 3A. FIG. 3A shows a test pattern observed indicating negative determination of Zika infection (anti-Zika antibody negative) in a patient sample using an assay cassette comprising a first lateral flow test strip (Anti-Zika Antibody Sandwich Assay Strip as in FIG. 1A) and a second lateral flow test strip (IgG strip as in FIG. 1B). A test signal at the second test line on the second test strip suggests infection with a related, non-Zika, pathogen. FIG. 3B. FIG. 3B shows a test pattern observed indicating negative determination of Zika infection (anti-Zika antibody negative) in a patient sample using an assay cassette comprising a first lateral flow test strip (Anti-Zika Antibody Sandwich Assay Strip as in FIG. 1A) and a second lateral flow test strip (IgG and IgM strip as in FIG. 1C). Test signals at the second and third test lines on the second test strip suggest infection with a related, non-Zika, pathogen. FIG. 3C. FIG. 3C shows a test pattern observed indicating negative determination of Zika infection (anti-Zika antibody negative) in a patient sample using an assay cassette comprising a first lateral flow test strip (Anti-Zika Antibody Sandwich Assay Strip as in FIG. 1A) and a second lateral flow test strip (IgG and IgM strip as in FIG. 1C).

FIGS. 4A to 4D. Determination of Intermediate or Late Zika Virus Infection Status—Test Pattern Examples. FIG. 4A. FIG. 4A shows a test pattern observed indicating determination of either intermediate or late Zika infection (anti-Zika antibody positive, anti-Zika IgG positive,) in a patient sample using an assay cassette comprising a first lateral flow test strip (Anti-Zika Antibody Sandwich Assay Strip as in FIG. 1A) and a second lateral flow test strip (IgG strip as in FIG. 1B). FIG. 4B. FIG. 4B shows a test pattern observed indicating determination of late Zika infection (anti-Zika antibody positive, anti-Zika IgG positive, IgM negative) in a patient sample using an assay cassette comprising a first lateral flow test strip (Anti-Zika Antibody Sandwich Assay Strip as in FIG. 1A) and a second lateral flow test strip (IgG and IgM strip as in FIG. 1C). FIG. 4C. FIG. 4C shows a test pattern observed indicating determination of intermediate Zika infection (anti-Zika antibody positive, anti-Zika IgG positive, anti-Zika IgM positive) in a patient sample using an assay cassette comprising a first lateral flow test strip (Anti-Zika Antibody Sandwich Assay Strip as in FIG. 1A) and a second lateral flow test strip (IgG and IgM strip as in FIG. 1C). FIG. 4D. FIG. 4D shows a test pattern observed indicating determination of late Zika infection (anti-Zika antibody positive, anti-Zika IgM negative) in a patient sample using an assay cassette comprising a first lateral flow test strip (Anti-Zika Antibody Sandwich Assay Strip as in FIG. 1A) and a second lateral flow test strip (IgM strip as in FIG. 1B).

FIG. 5. Immunological Response to Primary Dengue Infection. Solid line—Dengue viral protein, NS1; Dotted Line—Virus; Intermittently Dotted Line, DENV-reactive IgM; Dashed Line—DENV-reactive IgG. Figure reproduced from Laboratory Guidance and Diagnostic Testing, Dengue, Centers for Disease Control and Prevention, Atlanta, Ga., CDC website.

### DETAILED DESCRIPTION OF THE INVENTION

The present invention relates to immunochromatographic lateral flow assay compositions and methods useful for accurate and rapid determination of Zika virus infection status in patient samples. The invention combines two types of Zika tests, a highly specific anti-Zika antibody sandwich assay on a first lateral flow test strip, and an anti-Zika subtype antibody assay on a second lateral flow test strip. The analysis of the two test strips in combination enables the user to determine detailed infection status.

### Zika Virus

The Zika virus (ZIKV) is a member of the flaviviridae family. It has a single positive sense RNA genome that is initially translated as a single polyprotein which is cleaved post-transitionally into three structural proteins (C, PrM or M, and E) and seven nonstructural (NS) proteins (NS1, NS2A, NS2B, NS3, NS4A, NS4B and NS5)(1). The NS1 protein is a 352-amino-acid polypeptide that has a molecular weight of 46-55 kDa, depending on its glycosylation status. The glycosated NS1 protein forms a homodimer inside infected cells and is necessary for viral replication and late in infection (Suthar, et al., 2013, Nat. Rev. Microbiol. 11, 115-128, incorporated herein by reference). The NS1 protein can be secreted into the extracellular space as a hexameric lipoprotein particle, that assists the virus in immune evasion and pathogenesis by interacting with components from both innate and adaptive immune systems and other host factors (Song, et al., 2016, Nat. Struct. Mol. Biol. 23, 456-458, incorporated herein by reference). NS1 thus can serve as an important biomarker for early diagnosis of disease.

Test Principle

The first immunochromatographic lateral flow test strip (also referred to herein as “first test strip”) identifies an anti-Zika antibody in the patient sample by requiring binding of the antibody to each of two Zika antigen molecules in a highly specific sandwich assay format, to produce a test signal. The first test strip therefore confirms Zika virus infection, and has very low cross-reactivity with antibodies to other flaviviruses.

The second immunochromatographic lateral flow test strip (also referred to herein as “second test strip”) gives subtype information about the anti-Zika antibody, by requiring binding of an anti-Zika antibody in the patient sample to a Zika antigen molecule and an anti-human subtype antibody to produce a test signal. In embodiments, the second lateral flow test strip detects the presence of an IgG antibody, an IgM antibody, or both. When combined with a positive anti-Zika antibody signal on the first test strip, an anti-Zika antibody subtype identified on the second test strip can determine the Zika virus infection status of the patient, even in the absence of a test for the subtype antibody that indicates infection status.

### Determining Zika Virus Infection Status

The immunochromatographic lateral flow assay device and methods of the invention are used to determine Zika virus infection status using by detecting differences in anti-Zika antibody subtype expression levels following Zika infection and onset of symptoms.

Depending on the detection method used, Zika virus primary infection can show detectable IgM during the early stages of infection, starting as early as one day after onset of symptoms, detectable IgG together with IgM starting around 1-2 weeks after onset of symptoms and continuing for several weeks, and IgG without IgM detectable thereafter.

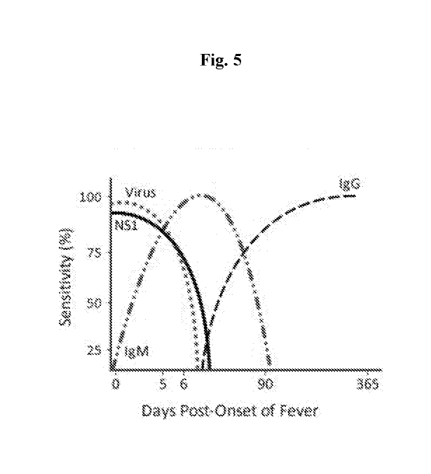

In embodiments, the timeline of immunological response to Zika virus infection is modeled according to that described for Dengue virus. For example, a graphical timeline of immunological response to a primary Dengue infection is illustrated by the Centers for Disease Control and Prevention, Atlanta, Ga. (see FIG. 5, reproduced from Laboratory Guidance and Diagnostic Testing, Dengue, Centers for Disease Control and Prevention, Atlanta, Ga., available on the CDC web site accessed Feb. 6, 2018, incorporated herein by reference). According to this guidance, a primary dengue infection is characterized by a slow and low titer antibody response, with IgM being the first immunoglobulin isotype to appear. Anti-dengue IgG is detectable at low titer at the end of the first week of illness, and slowly increases.

The present invention provides assay compositions and methods that accurately and quickly detect the presence of anti-Zika IgM and/or anti-Zika IgG antibody subtypes in individuals with suspected infection, thereby determining Zika virus infection status. This temporal stage of infection reflects contagiousness of disease, with higher virus transmission rates typically seen during IgM production stages (i.e., in patients having early and intermediate infection status). In embodiments, the immunochromatographic lateral flow assay compositions and methods of the invention are used to determine early Zika virus infection (IgG not detected, IgM detected or inferred), intermediate Zika virus infection (IgM detected, IgG detected), and late Zika virus infection (IgM not detected, IgG detected or inferred). In embodiments, the confirmation of Zika virus infection and determination of Zika virus infection status is confirmed using one or more confirmatory assay. For example, in embodiments, the presence of anti-Zika IgM and/or IgG is confirmed by ELISA. In embodiments, virus infection is confirmed by PCR testing of viral nucleic acids. It is noted that no other available assay, including the more cumbersome ELISA and PCR assays, provides 100% sensitivity or specificity. Rapid antibody subtype assays often show high levels of cross-reactivity.

### Determination of Early Zika Virus Infection Status Using the Invention

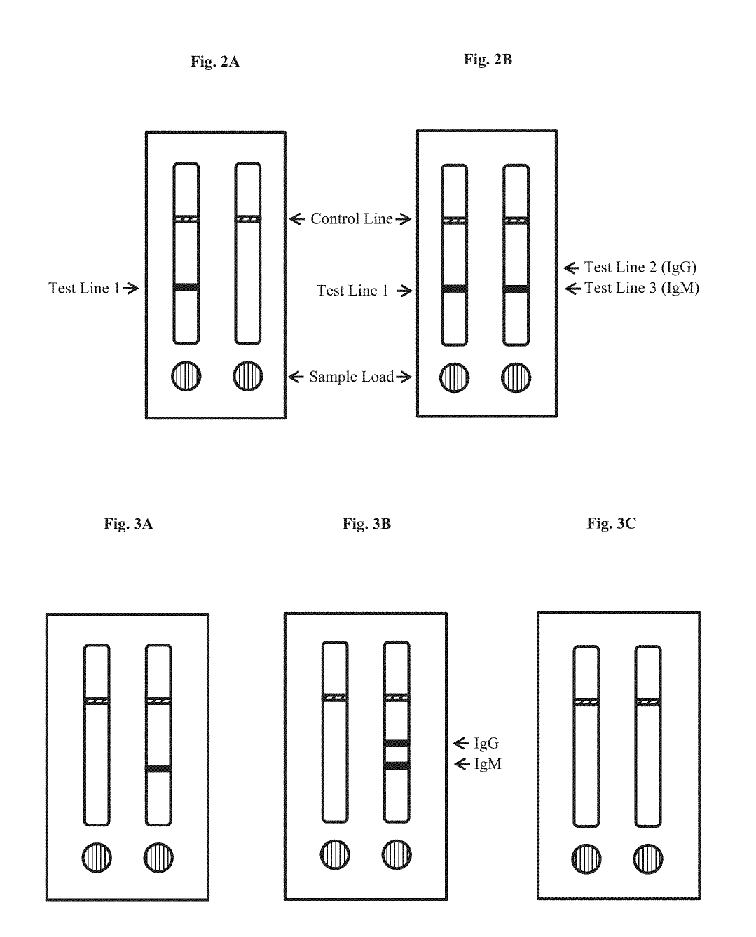

In embodiments, determination of early Zika virus infection status, characterized by the prexence of anti-Zika IgM and the absence of anti-Zika IgG, is made using an assay of the present invention comprising a second test strip that comprises an anti-Zika IgG test and no IgM test. This determination is illustrated, for example, in FIG. 2A, which shows the presence of an anti-Zika antibody on the first test strip at the first test line (Test Line 1), and no detection of anti-Zika IgG on the second test strip at the second test line (Test Line 2). Taken together, these two results indicate the presence of anti-Zika antibody that is not IgG. The presence of IgM is deduced from the results. (The figures also show a signal at the control line, representing successful flow of the tracer reagent to the control line and indicating that the test strip operated as intended.)

In embodiments, an assay that includes an IgM test but no IgG test is not used to determine early Zika virus infection, as it does not indicate the absence of IgG. In embodiments, early Zika virus infection status is determined using an assay comprising a second test strip that includes both anti-Zika IgG and IgM tests. FIG. 2B shows an example of an early Zika virus infection test pattern observed, wherein the test signal indicates the presence of anti-Zika IgM and the absence of anti-Zika IgG. In embodiments, early Zika virus infection status is determined using an assay comprising a first sandwich assay test strip and two antibody subtype test strips, one that tests anti-Zika IgG and one that tests anti-Zika IgM.

Accordingly, in embodiments, an early Zika virus infection status is determined in a patient sample according to the invention wherein the first test strip indicates the presence of an anti-Zika antibody, and the second test strip indicates the presence of an anti-Zika antibody that is IgM and the absence of an anti-Zika antibody that is IgG, based on one of the following results:

a) where only anti-Zika IgG is tested on the second test strip, anti-Zika IgG is not detected;

b) where IgG and IgM are tested on the second test strip, anti-Zika IgG is not detected and anti-Zika IgM is detected.

In embodiments, a control line signal is further observed on each of the first and second test strips.

In embodiments, a determination of early Zika virus infection status in a patient sample having detectable anti-Zika antibody is made when anti-Zika IgM is detectable and anti-Zika IgG is not detectable, in a test for anti-Zika antibody, anti-Zika IgM, and anti-Zika IgG. In embodiments, a determination of early Zika virus infection status is made in a patient sample having detectable anti-Zika antibody when anti-Zika IgG is not detectable, in a test for anti-Zika antibody and anti-Zika IgG.

In embodiments, early Zika virus infection is defined as a period ranging from about 1 to about 12 days post-onset of the earliest observed Zika disease symptom. In embodiments, this period is about 1 day to about 12 days. In embodiments, this period is at least about 1 day. In embodiments, this period is at most about 12 days. In embodiments, this period is about 1 day to about 2 days, about 1 day to about 3 days, about 1 day to about 4 days, about 1 day to about 5 days, about 1 day to about 6 days, about 1 day to about 7 days, about 1 day to about 8 days, about 1 day to about 9 days, about 1 day to about 10 days, about 1 day to about 11 days, about 1 day to about 12 days, about 2 days to about 3 days, about 2 days to about 4 days, about 2 days to about 5 days, about 2 days to about 6 days, about 2 days to about 7 days, about 2 days to about 8 days, about 2 days to about 9 days, about 2 days to about 10 days, about 2 days to about 11 days, about 2 days to about 12 days, about 3 days to about 4 days, about 3 days to about 5 days, about 3 days to about 6 days, about 3 days to about 7 days, about 3 days to about 8 days, about 3 days to about 9 days, about 3 days to about 10 days, about 3 days to about 11 days, about 3 days to about 12 days, about 4 days to about 5 days, about 4 days to about 6 days, about 4 days to about 7 days, about 4 days to about 8 days, about 4 days to about 9 days, about 4 days to about 10 days, about 4 days to about 11 days, about 4 days to about 12 days, about 5 days to about 6 days, about 5 days to about 7 days, about 5 days to about 8 days, about 5 days to about 9 days, about 5 days to about 10 days, about 5 days to about 11 days, about 5 days to about 12 days, about 6 days to about 7 days, about 6 days to about 8 days, about 6 days to about 9 days, about 6 days to about 10 days, about 6 days to about 11 days, about 6 days to about 12 days, about 7 days to about 8 days, about 7 days to about 9 days, about 7 days to about 10 days, about 7 days to about 11 days, about 7 days to about 12 days, about 8 days to about 9 days, about 8 days to about 10 days, about 8 days to about 11 days, about 8 days to about 12 days, about 9 days to about 10 days, about 9 days to about 11 days, about 9 days to about 12 days, about 10 days to about 11 days, about 10 days to about 12 days, or about 11 days to about 12 days.

An overview of Zika virus infection is provided by the Centers for Disease Control and Prevention, Atlanta, Ga. (CDC website, Zika Virus Home, accessed Feb. 6, 2008), and Zika testing is described in, e.g., “Updated Guidance for US Laboratories Testing for Zika Virus Infection,” Jul. 24, 2017, Centers for Disease Control and Prevention, Atlanta, Ga., both incorporated herein by reference in their entirety. According to the CDC, the most common symptoms of Zika virus infection include fever, rash, joint pain, muscle pain, and conjunctivitis.

### Determination of Intermediate Zika Virus Infection Status Using the Invention

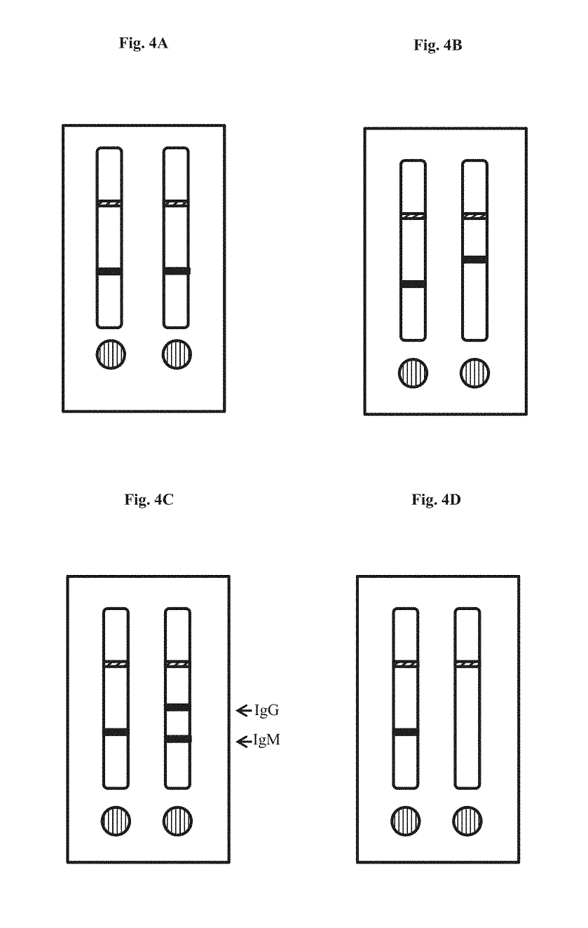

In embodiments, intermediate Zika virus infection status, characterized by the presence of IgG both anti-Zika IgM and IgG, is determined by an assay of the invention using a second test strip comprising both an anti-Zika IgG test and an anti-Zika IgM test (e.g., FIG. 4C). In embodiments, an assay that comprises only one of an IgG test or an IgM test does is not used to determine intermediate Zika virus infection, as it does not indicate the presence of both IgG and IgM (see, e.g., FIG. 4A).

In embodiments, an intermediate Zika virus infection status is determined in a patient sample using an assay of the invention wherein the first test strip indicates the presence of an anti-Zika antibody, and where both anti-Zika IgM and IgG are tested, the second test strip indicates the presence of both anti-Zika IgM and IgG antibodies.

In embodiments, a control line signal is further observed on each of the first and second test strips.

In embodiments, a determination of intermediate Zika virus infection status in a patient sample having detectable anti-Zika antibody is made when anti-Zika IgM and anti-Zika IgG are detectable, in a test for anti-Zika antibody, anti-Zika IgM, and anti-Zika IgG.

In embodiments, intermediate Zika virus infection is defined as a period ranging from about 10 to about 90 days post-onset of the earliest observed Zika disease symptom. In embodiments, this period is about 10 days to about 90 days. In embodiments, this period is at least about 10 days. In embodiments, this period is at most about 90 days. In embodiments, this period is about 10 days to about 20 days, about 10 days to about 30 days, about 10 days to about 40 days, about 10 days to about 50 days, about 10 days to about 60 days, about 10 days to about 70 days, about 10 days to about 80 days, about 10 days to about 90 days, about 20 days to about 30 days, about 20 days to about 40 days, about 20 days to about 50 days, about 20 days to about 60 days, about 20 days to about 70 days, about 20 days to about 80 days, about 20 days to about 90 days, about 30 days to about 40 days, about 30 days to about 50 days, about 30 days to about 60 days, about 30 days to about 70 days, about 30 days to about 80 days, about 30 days to about 90 days, about 40 days to about 50 days, about 40 days to about 60 days, about 40 days to about 70 days, about 40 days to about 80 days, about 40 days to about 90 days, about 50 days to about 60 days, about 50 days to about 70 days, about 50 days to about 80 days, about 50 days to about 90 days, about 60 days to about 70 days, about 60 days to about 80 days, about 60 days to about 90 days, about 70 days to about 80 days, about 70 days to about 90 days, or about 80 days to about 90 days. In embodiments, this period is about 11 days to about 60 days. In embodiments, this period is at least about 11 days. In embodiments, this period is at most about 60 days. In embodiments, this period is about 11 days to about 15 days, about 11 days to about 20 days, about 11 days to about 25 days, about 11 days to about 30 days, about 11 days to about 35 days, about 11 days to about 40 days, about 11 days to about 45 days, about 11 days to about 50 days, about 11 days to about 55 days, about 11 days to about 60 days, about 15 days to about 20 days, about 15 days to about 25 days, about 15 days to about 30 days, about 15 days to about 35 days, about 15 days to about 40 days, about 15 days to about 45 days, about 15 days to about 50 days, about 15 days to about 55 days, about 15 days to about 60 days, about 20 days to about 25 days, about 20 days to about 30 days, about 20 days to about 35 days, about 20 days to about 40 days, about 20 days to about 45 days, about 20 days to about 50 days, about 20 days to about 55 days, about 20 days to about 60 days, about 25 days to about 30 days, about 25 days to about 35 days, about 25 days to about 40 days, about 25 days to about 45 days, about 25 days to about 50 days, about 25 days to about 55 days, about 25 days to about 60 days, about 30 days to about 35 days, about 30 days to about 40 days, about 30 days to about 45 days, about 30 days to about 50 days, about 30 days to about 55 days, about 30 days to about 60 days, about 35 days to about 40 days, about 35 days to about 45 days, about 35 days to about 50 days, about 35 days to about 55 days, about 35 days to about 60 days, about 40 days to about 45 days, about 40 days to about 50 days, about 40 days to about 55 days, about 40 days to about 60 days, about 45 days to about 50 days, about 45 days to about 55 days, about 45 days to about 60 days, about 50 days to about 55 days, about 50 days to about 60 days, or about 55 days to about 60 days.

### Determination of Late Zika Virus Infection Status Using the Invention

In embodiments, a determination of late Zika virus infection status, characterized by the presence of anti-Zika IgG and the absence of anti-Zika IgM, is determined using an assay of the present invention, wherein the second test strip comprises an anti-Zika IgM test and no IgG test. This determination is illustrated, for example, in FIG. 4D, which shows the presence of an anti-Zika antibody test signal on the first test strip at the first test line, and the absence of an anti-Zika IgM test signal on the second test strip at the second test line. Taken together, these two results indicate the presence of anti-Zika antibody that is not IgM. The presence of IgG is deduced from the results.

In embodiments, an assay that includes an IgG test but no IgM test is not used to determine late Zika virus infection status, as it does not indicate the absence of IgM (see, e.g., FIG. 4A). In embodiments, late Zika virus infection status is determined using an assay comprising a second test strip that includes both anti-Zika IgG and IgM tests (e.g., FIG. 4B). FIG. 4B shows an example of a late Zika virus infection test pattern, wherein test signal is present at the IgG test line but not at the IgM test line.

Therefore, in embodiments, a late Zika virus infection status is determined in a patient sample according to the invention wherein the first test strip indicates the presence of an anti-Zika antibody, and the second test strip indicates the presence of an anti-Zika antibody that is IgG and the absence of an antibody that is IgM, based on one of the following results:

a) where only IgM is tested on the second test strip, anti-Zika IgM is not detected;

b) where anti-Zika IgG and IgM are tested on the second test strip, anti-Zika IgG is detected and anti-Zika IgM is not detected.

In embodiments, a control line signal is further observed on each of the first and second test strips.

In embodiments, a determination of late Zika virus infection status in a patient sample having detectable anti-Zika antibody is made when anti-Zika IgG is detectable and anti-Zika IgM is not detectable, in a test for anti-Zika antibody, anti-Zika IgM, and anti-Zika IgG. In embodiments, a determination of late Zika virus infection status is made in a patient sample having detectable anti-Zika antibody when anti-Zika IgM is not detectable, in a test for anti-Zika antibody and anti-Zika IgM.

In embodiments, late Zika virus infection is defined as a period greater than about 60 days to greater than about 90 days post-onset of the earliest observed Zika disease symptom. In embodiments, this period is greater than about 60 days, about 65 days, about 70 days, about 75 days, about 80 days, about 85 days, or about 90 days. In embodiments, this period is greater than about about 60 days to about 65 days, about 60 days to about 70 days, about 60 days to about 75 days, about 60 days to about 80 days, about 60 days to about 85 days, about 60 days to about 90 days, about 65 days to about 70 days, about 65 days to about 75 days, about 65 days to about 80 days, about 65 days to about 85 days, about 65 days to about 90 days, about 70 days to about 75 days, about 70 days to about 80 days, about 70 days to about 85 days, about 70 days to about 90 days, about 75 days to about 80 days, about 75 days to about 85 days, about 75 days to about 90 days, about 80 days to about 85 days, about 80 days to about 90 days, or about 85 days to about 90 days.

### Determination of No Zika Virus Infection Status or No Zika Virus Infection Status and Non-Zika Virus Infection Status Using the Invention

In embodiments, a no Zika virus infection status is determined in a patient sample according to the invention wherein the first test strip indicates the absence of an anti-Zika antibody. In these embodiments the second lateral flow test strip can indicate the presence or absence of an anti-Zika antibody that is IgM or IgG, where the second lateral flow test strip tests IgG, IgM, or both IgG and IgM. Thus, the negative anti-Zika antibody result, combined with any antibody subtype test result, indicates no infection with Zika virus.

In embodiments, both a no Zika virus infection and a non-Zika virus infection status (infection with a non-Zika virus) is determined in a patient sample according to the invention wherein the first test strip indicates the absence of an anti-Zika antibody, and the second lateral flow test strip indicates the presence of an anti-Zika antibody that is IgM, IgG, or both, wherein the second lateral flow test strip tests IgG, IgM, or both IgG and IgM, respectively. Due to the high specificity of the sandwich assay on the first test strip, the absence of a Zika antibody indicated by the first test strip is a reliable indication that Zika antibodies are not present. The anti-human subtype antibody assay on the second test strip is less specific, therefore, in view of the first test strip result, the presence of an anti-Zika subtype antibody indicated by the second test strip can be concluded to result from cross-reactivity of a subtype antibody assay on the second test strip with a non-Zika antibody, e.g., an anti-Dengue antibody.

In embodiments, the first test strip indicates the absence of an anti-Zika antibody when the patient sample does not comprise anti-Zika antibodies, and does comprise an antibody specific for a different infective agent or pathogen. In embodiments, the different infective agent is a flavivirus. In embodiments, the different infective agent is selected from: an anti-Dengue antibody, an anti-Chikungunya antibody, an anti-Yellow Fever antibody, an anti-Malaria antibody, and an anti-West Nile virus antibody.

In embodiments, a control line signal is further observed on each of the first and second test strips.

### Immunochromatographic Lateral Flow Assay, Device and Reagents

The invention thus relates to a two-strip immunochromatographic lateral flow assay for determining Zika Virus infection status in a patient, an assay device, methods for use of the assay and device, and kits comprising the immunochromatographic lateral flow assay and device.

The reagents and components of each of the first lateral flow test strip and the second lateral flow test strip (also referred to as “first test strip” and “second test strip”) can be described by dividing them into test strip zones. Using the guidance provided herein, the knowledge of one of skill in the art and the published literature, reagents for use in each zone can be selected and incorporated into the assay, and the assay operated based on individual needs, available resources, etc, as appropriate. The general principles of immunochromatographic lateral flow assays, and standard elements of lateral flow test strips, are well known in the art and described in the literature, e.g., in: “Rapid Lateral Flow Test Strips. Considerations for Product Development,” EMD Millipore 2013; Koczula, et al., 2016, “Lateral flow assays,” Essays in Biochemistry 60: 111-120; Sharma, et al., 2015, “Point-of-Care Diagnostics in Low Resource Settings: Present Status and Future Role of Microfluidics,” Biosensors 5: 577-601; Sajid, et al., 2014, “Designs, formats and applications of lateral flow assay: A literature review,” J. Saudi Chem. Soc. 19:689-705; Holstein, et al., “Immobilizing affinity proteins to nitrocellulose: a toolbox for paper-based assay developers,” 2016, Analytical and Bioanalytical Chemistry 408(5):1335-46; U.S. Pat. No. 9,034,657, “Two step lateral flow assay methods and devices;” U.S. Pat. Nos. 4,313,734; 4,376,110; 4,435,504; 4,703,017; 4,855,240; 4,954,452; 5,028,535; 5,075,078; 5,654,162; WO 95/16207; EP 0810436; and U.S. Pat. No. 8,859,265, “Lateral flow immunoassay device with a more rapid and accurate test result;” each incorporated by reference herein in its entirety.

In embodiments, the first and second test strips comprise zones that include, but are not limited to, Zones 1 to 5, wherein sample applied at Zone 1 flows toward Zone 5, as described below.

### Test Strips

### Zone 1: Sample Loading Pad

The sample loading pad is provided to accept the sample to be analyzed, e.g., a patient body fluid sample suspected of containing a target analyte, e.g., an anti-Zika antibody. The loading pad can be made from, e.g., cellulose, glass fiber or any other appropriate material known in the art. The sample is applied to the loading pad, followed by application of a chase buffer, e.g., PBS, where it is transferred by capillary action through the loading pad toward Zone 2.

The chase buffer enables proper flow of the tracer reagents on each of the first and second test strips to the control line on each test strip, and enables the flow of a complex comprising the tracer reagent on each of the first and second test strips, to each respective test line on each test strip.

### Zone 2: Conjugate Pad

The conjugate pad (conjugate release pad) of each of the first and second test strips comprises a labeled tracer reagent, dried to the pad. The conjugate pad can be made from, e.g., a non-absorbent material such as fiberglass pad, polyester, rayon or another appropriate material as known in the art. Typically, when the conjugate pad is contacted with the sample and the chase buffer, the tracer reagent (mobile phase) is solubilized, mixes with the sample, and flows from the conjugate pad into the test strip material toward Zone 3.

The tracer reagent on the conjugate pad is selected to bind specifically to the target analyte in the sample to form a tracer reagent—target analyte complex. In embodiments, the target analyte is an anti-Zika antibody in the sample that reacts with a Zika tracer reagent that is a labeled Zika antigen (“first Zika tracer antigen” on first test strip; “second Zika tracer antigen” on second test strip) on the conjugate pad, forming a Zika tracer antigen—anti-Zika antibody complex. In embodiments, the tracer reagent of either the first and second test strip is a Zika antigen selected from: recombinant Zika NS1, a fragment of recombinant Zika NS1, recombinant Zika ENV, a fragment of recombinant Zika ENV, recombinant Zika VLP, a fragment of recombinant Zika VLP, and purified Zika Virus Lysate. The tracer reagent, or any tracer reagent described herein, can be labeled with any appropriate labeling agent known in the art, e.g., with enzymes, fluorescent tags, gold nanoparticles, quantum dots, or latex beads, using a suitable method as known in the art and described in the literature. Labeled tracer reagents useful in the compositions and methods of the present invention can be readily obtained from commercial sources. In particular embodiments, the tracer reagent is labeled with gold nanoparticles.

### First Lateral Flow Test Strip Conjugate Pad

In embodiments, the target analyte is an anti-Zika antibody in the sample that recognizes the the first Zika tracer antigen on the first lateral flow test strip conjugate pad, forming a complex comprising the first Zika tracer antigen—anti-Zika antibody.

### Second Lateral Flow Test Strip Conjugate Pad

In embodiments, the second tracer reagent, on the conjugate pad of the second lateral flow test strip, is a Zika antigen (second Zika tracer antigen). In other embodiments, the second tracer reagent is an anti-human subtype tracer antibody.

In embodiments, the second Zika tracer antigen specifically binds to an anti-Zika antibody in the sample. This forms a complex comprising the second Zika tracer antigen—anti-Zika antibody.

In certain embodiments, wherein the second lateral flow test strip tests the presence of one of IgG, IgM, or IgA antibody in a sample, the second tracer reagent can be an anti-human subtype tracer antibody that specifically binds to an antibody in the sample that is one of: IgG, IgM, and IgA. In these embodiments, the anti-Zika antibody in the sample binds to the anti-human subtype antibody on the second lateral flow test strip conjugate pad to form a complex comprising the anti-human subtype tracer antibody—anti-Zika antibody.

### Test Line Zone 3 (Detection Zone)

Each of the first test strip and second test strips comprises an immobilized detection reagent (“first immobilized detection reagent,” and “second immobilized detection reagent,” respectively) at a test line in Zone 3 (“first test line,” of the first test strip, and “second test line,” and “third test line,” of the first second strip which can comprise either one or two test lines, as described). The immobilized detection reagent binds to the tracer reagent—target analyte complex.

### First Test Line Zone 3—First Immobilized Detection Reagent

In embodiments, the first test strip is an anti-Zika antibody sandwich assay strip, wherein the first tracer reagent on the conjugate pad, and the first immobilized detection reagent at the first test line, both are Zika antigens that bind to an anti-Zika antibody potentially present in the patient sample. In embodiments, they are the same Zika antigen. In embodiments, the first immobilized detection reagent is a Zika virus antigen selected from, e.g.: a recombinant Zika NS1, a fragment of recombinant Zika NS1, recombinant Zika ENV, a fragment of recombinant Zika ENV, recombinant Zika VLP, a fragment of recombinant Zika VLP, and purified Zika Virus Lysate, and is referred to as the first immobilized Zika detection antigen.

In embodiments, the first immobilized Zika detection antigen binds to the anti-Zika antibody of the first Zika tracer antigen—anti-Zika antibody complex from Zone 2, thereby forming an immobilized complex comprising the first Zika tracer antigen—anti-Zika antibody—first immobilized Zika detection antigen. This stationary complex produces a test signal at the first test line indicative of the presence of the anti-Zika antibody in the sample.

### Second Test Line Zone 3A—Second Immobilized Detection Reagent

In embodiments, the second lateral flow test strip is an anti-Zika antibody subtype assay test strip, wherein the second tracer reagent is a Zika antigen, and the second immobilized detection reagent is an anti-human subtype antibody that is specific for subtype IgG, IgM, or IgA (“second test line immobilized detection antibody”). In embodiments, the anti-human subtype antibody is a monoclonal antibody. In embodiments, the second test line immobilized detection antibody binds to the anti-Zika antibody of the second Zika tracer antigen—anti-Zika antibody complex from Zone 2, thereby forming an immobilized complex comprising the second Zika tracer antigen—anti-Zika antibody—second test line immobilized Zika detection antibody. This stationary complex produces a test signal at the second test line indicative of the presence of the anti-Zika subtype antibody tested in the sample.

In other embodiments, wherein the second lateral flow test strip tests the presence of one of IgG, IgM, or IgA antibody in a sample, the second tracer reagent is an anti-human subtype tracer antibody that specifically binds to an antibody in the sample that is one of: IgG, IgM, and IgA. Thus, the anti-Zika antibody in the sample binds to the anti-human subtype antibody on the second lateral flow test strip conjugate pad to form a complex comprising the anti-human subtype tracer antibody—anti-Zika antibody. In these embodiments, the second immobilized detection reagent is instead a Zika virus antigen, e.g.: a recombinant Zika NS1, a fragment of recombinant Zika NS1, recombinant Zika ENV, a fragment of recombinant Zika ENV, recombinant Zika VLP, a fragment of recombinant Zika VLP, and purified Zika Virus Lysate, and is referred to as the second test line immobilized Zika detection antigen. In these embodiments, the complex formed at Zone 3A is anti-human subtype tracer antibody—anti-Zika antibody—second test line immobilized Zika detection antigen.

### Third Test Line Zone 3B—Third Immobilized Detection Reagent

In embodiments, the second lateral flow test strip is an anti-Zika antibody subtype assay that tests for two different antibody subtypes. In embodiments, the second tracer reagent is a Zika antigen, the second immobilized detection reagent is an anti-human subtype antibody that is specific for subtype IgG, IgM, or IgA, and the third immobilized detection reagent is an anti-human subtype antibody that is specific for subtype IgG, IgM, or IgA (“third test line immobilized detection antibody”), wherein the third test line immobilized detection antibody recognizes a different antibody subtype from that recognized by the second test line immobilized detection antibody. In embodiments, the anti-human subtype antibody is a monoclonal antibody. In embodiments, the third test line immobilized detection antibody binds to the anti-Zika antibody of the second Zika tracer antigen—anti-Zika antibody complex from Zone 2, thereby forming an immobilized complex comprising the second Zika tracer antigen—anti-Zika antibody—third test line immobilized Zika detection antibody. This stationary complex produces a test signal at the third test line indicative of the presence of the anti-Zika subtype antibody tested in the sample.

In other embodiments, wherein the second lateral flow test strip tests the presence of one of IgG, IgM, or IgA antibody in a sample, the second tracer reagent is an anti-human subtype tracer antibody that specifically binds to an antibody in the sample that is one of: IgG, IgM, and IgA. Thus, the anti-Zika antibody in the sample binds to the anti-human tracer subtype antibody on the second lateral flow test strip conjugate pad to form a complex comprising the anti-human subtype tracer antibody—anti-Zika antibody. In these embodiments, the third immobilized detection reagent is instead a Zika virus antigen, e.g.: a recombinant Zika NS1, a fragment of recombinant Zika NS1, recombinant Zika ENV, a fragment of recombinant Zika ENV, recombinant Zika VLP, a fragment of recombinant Zika VLP, and purified Zika Virus Lysate, and is referred to as the third test line immobilized Zika detection antigen. In these embodiments, the complex formed at Zone 3B is anti-human subtype tracer antibody—anti-Zika antibody—third test line immobilized Zika detection antigen.

### Zone 4—Control Line

In embodiments, a control line is included on each test strip to indicate proper operation of the assay. In embodiments, the presence of a control signal at the control line is intended to indicate successful flow of the labeled tracer reagent across the test strip, e.g., from Zone 2 to Zone 4, and that binding of tracer reagent to a control line reagent at Zone 4 occurs. In embodiments, the control line comprises an immobilized control line reagent that can specifically bind to the tracer reagent of Zone 2. In embodiments wherein the tracer reagent is a Zika antigen, the control line comprises an immobilized anti-Zika antibody, e.g., an anti-Zika antigen monoclonal antibody, which binds specifically to the Zika antigen used as the tracer reagent as described above in relation to Zone 2. In embodiments, the tracer reagent is Zika NS1, and the control line reagent is a Zika NS1 mAb. For example, monoclonal antibodies specific for Zika antigens, e.g., ENV and NS1, are commercially available, labeled and unlabeled, from many sources, e.g., from BioFront Technologies, Tallahassee, Fla.

In other embodiments, a second reagent (e.g., a non-Zika reagent), is labeled and conjugated to Zone 2 in mixture with the specific tracer reagent (e.g., Zika antigen). In these embodiments, the control line comprises an immobilized reagent that binds specifically to the labeled non-specific reagent. For example, a labeled non-Zika mouse antibody can be conjugated to Zone 2, and detected by an immobilized anti-mouse antibody when it reaches the Zone 4 control line.

### Zone 5—Absorbent Pad

An absorbent pad, e.g., made from cellulose filter, is included at the end of each strip. The absorbent pad can wick excess reagents and prevent backflow and fluid buildup on the strip.

### Sample

The compositions and methods of the present invention are useful for rapidly determining Zika virus infection status in a potentially infected individual by assaying a sample material (a patient sample). It is understood that the Zika virus infection status in the patient is determined by analyzing a patient sample, that is, determination of a patient's Zika virus infection status is made based on assay of the patient sample. In embodiments, the compositions and methods of the invention allow collection and testing of the sample material at home, by the patient or another individual, or by a health practitioner in a health care setting. In embodiments, the sample material is any body fluid sample suspected of comprising target analyte, e.g., an anti-Zika antibody, at levels detectable by the assay methods. In embodiments, the sample material and volume used are known to provide a test result with at least about 95% confidence or accuracy.

For example, the sample can comprise a body fluid selected from whole blood, plasma, serum, saliva, urine, sweat, tears, semen, and vaginal fluid. In specific embodiments, the sample comprises whole blood, plasma, serum, or saliva.

A small volume of sample is required to practice the methods of the invention. The appropriate volume to apply to the sample loading pad will be known to one of skill in the art of constructing immunochromatographic lateral flow assays (also known in the art as lateral flow immunoassays). In embodiments, a suitable amount of whole blood applied to the sample loading pad of one strip is about 5 uL to about 50 uL. In embodiments, a suitable amount of whole blood applied to the sample loading pad of one strip is at least about 5 uL. In embodiments, a suitable amount of whole blood applied to the sample loading pad of one strip is at most about 50 uL. In embodiments, a suitable amount of whole blood applied to the sample loading pad of one strip is about 5 uL, 10 uL, 15 uL, 20 uL, 25 uL, 30 uL, 35 uL, 40 uL, 45 uL, or 50 uL. In embodiments, a suitable amount of whole blood applied to the sample loading pad of one strip is about 5 uL to about 10 uL, about 5 uL to about 15 uL, about 5 uL to about 20 uL, about 5 uL to about 25 uL, about 5 uL to about 30 uL, about 5 uL to about 25 uL, about 5 uL to about 40 uL, about 5 uL to about 45 uL, about 5 uL to about 50 uL, about 10 uL to about 15 uL, about 10 uL to about 20 uL, about 10 uL to about 25 uL, about 10 uL to about 30 uL, about 10 uL to about 25 uL, about 10 uL to about 40 uL, about 10 uL to about 45 uL, about 10 uL to about 50 uL, about 15 uL to about 20 uL, about 15 uL to about 25 uL, about 15 uL to about 30 uL, about 15 uL to about 25 uL, about 15 uL to about 40 uL, about 15 uL to about 45 uL, about 15 uL to about 50 uL, about 20 uL to about 25 uL, about 20 uL to about 30 uL, about 20 uL to about 25 uL, about 20 uL to about 40 uL, about 20 uL to about 45 uL, about 20 uL to about 50 uL, about 25 uL to about 30 uL, about 25 uL to about 25 uL, about 25 uL to about 40 uL, about 25 uL to about 45 uL, about 25 uL to about 50 uL, about 30 uL to about 25 uL, about 30 uL to about 40 uL, about 30 uL to about 45 uL, about 30 uL to about 50 uL, about 25 uL to about 40 uL, about 25 uL to about 45 uL, about 25 uL to about 50 uL, about 40 uL to about 45 uL, about 40 uL to about 50 uL, or about 45 uL to about 50 uL.

In embodiments, a suitable amount of serum or plasma applied to the sample loading pad of one strip is about 3 uL to about 30 uL. In embodiments, a suitable amount of serum or plasma applied to the sample loading pad of one strip is at least about 3 uL. In embodiments, a suitable amount of serum or plasma applied to the sample loading pad of one strip is at most about 30 uL. In embodiments, a suitable amount of serum or plasma applied to the sample loading pad of one strip is about 2 uL, 3 uL, 4 uL, 5 uL, 6 uL, 7 uL, 8 uL, 9 uL, 10 uL, 11 uL, 12 uL, 13 uL, 14 uL, 15 uL, 16 uL, 17 uL, 18 uL, 19 uL, 20 uL, 25 uL, 30 uL, 35 uL, 40 uL, 45 uL, or 50 uL. In embodiments, a suitable amount of serum or plasma applied to the sample loading pad of one strip is about 3 uL to about 5 uL, about 3 uL to about 8 uL, about 3 uL to about 10 uL, about 3 uL to about 13 uL, about 3 uL to about 15 uL, about 3 uL to about 18 uL, about 3 uL to about 20 uL, about 3 uL to about 23 uL, about 3 uL to about 25 uL, about 3 uL to about 28 uL, about 3 uL to about 30 uL, about 5 uL to about 8 uL, about 5 uL to about 10 uL, about 5 uL to about 13 uL, about 5 uL to about 15 uL, about 5 uL to about 18 uL, about 5 uL to about 20 uL, about 5 uL to about 23 uL, about 5 uL to about 25 uL, about 5 uL to about 28 uL, about 5 uL to about 30 uL, about 8 uL to about 10 uL, about 8 uL to about 13 uL, about 8 uL to about 15 uL, about 8 uL to about 18 uL, about 8 uL to about 20 uL, about 8 uL to about 23 uL, about 8 uL to about 25 uL, about 8 uL to about 28 uL, about 8 uL to about 30 uL, about 10 uL to about 13 uL, about 10 uL to about 15 uL, about 10 uL to about 18 uL, about 10 uL to about 20 uL, about 10 uL to about 23 uL, about 10 uL to about 25 uL, about 10 uL to about 28 uL, about 10 uL to about 30 uL, about 13 uL to about 15 uL, about 13 uL to about 18 uL, about 13 uL to about 20 uL, about 13 uL to about 23 uL, about 13 uL to about 25 uL, about 13 uL to about 28 uL, about 13 uL to about 30 uL, about 15 uL to about 18 uL, about 15 uL to about 20 uL, about 15 uL to about 23 uL, about 15 uL to about 25 uL, about 15 uL to about 28 uL, about 15 uL to about 30 uL, about 18 uL to about 20 uL, about 18 uL to about 23 uL, about 18 uL to about 25 uL, about 18 uL to about 28 uL, about 18 uL to about 30 uL, about 20 uL to about 23 uL, about 20 uL to about 25 uL, about 20 uL to about 28 uL, about 20 uL to about 30 uL, about 23 uL to about 25 uL, about 23 uL to about 28 uL, about 23 uL to about 30 uL, about 25 uL to about 28 uL, about 25 uL to about 30 uL, or about 28 uL to about 30 uL.

In embodiments, a suitable amount of saliva applied to the sample loading pad of one strip is about 5 uL to about 50 uL. In embodiments, a suitable amount of saliva applied to the sample loading pad of one strip is at least about 5 uL. In embodiments, a suitable amount of saliva applied to the sample loading pad of one strip is at most about 50 uL. In embodiments, a suitable amount of saliva applied to the sample loading pad of one strip is about 5 uL, 10 uL, 15 uL, 20 uL, 25 uL, 30 uL, 35 uL, 40 uL, 45 uL, or 50 uL. In embodiments, a suitable amount of saliva applied to the sample loading pad of one strip is about 5 uL to about 10 uL, about 5 uL to about 15 uL, about 5 uL to about 20 uL, about 5 uL to about 25 uL, about 5 uL to about 30 uL, about 5 uL to about 25 uL, about 5 uL to about 40 uL, about 5 uL to about 45 uL, about 5 uL to about 50 uL, about 10 uL to about 15 uL, about 10 uL to about 20 uL, about 10 uL to about 25 uL, about 10 uL to about 30 uL, about 10 uL to about 25 uL, about 10 uL to about 40 uL, about 10 uL to about 45 uL, about 10 uL to about 50 uL, about 15 uL to about 20 uL, about 15 uL to about 25 uL, about 15 uL to about 30 uL, about 15 uL to about 25 uL, about 15 uL to about 40 uL, about 15 uL to about 45 uL, about 15 uL to about 50 uL, about 20 uL to about 25 uL, about 20 uL to about 30 uL, about 20 uL to about 25 uL, about 20 uL to about 40 uL, about 20 uL to about 45 uL, about 20 uL to about 50 uL, about 25 uL to about 30 uL, about 25 uL to about 25 uL, about 25 uL to about 40 uL, about 25 uL to about 45 uL, about 25 uL to about 50 uL, about 30 uL to about 25 uL, about 30 uL to about 40 uL, about 30 uL to about 45 uL, about 30 uL to about 50 uL, about 25 uL to about 40 uL, about 25 uL to about 45 uL, about 25 uL to about 50 uL, about 40 uL to about 45 uL, about 40 uL to about 50 uL, or about 45 uL to about 50 uL.

In embodiments, a suitable amount of semen applied to the sample loading pad of one strip is about 5 uL to about 50 uL. In embodiments, a suitable amount of semen applied to the sample loading pad of one strip is at least about 5 uL. In embodiments, a suitable amount of semen applied to the sample loading pad of one strip is at most about 50 uL. In embodiments, a suitable amount of semen applied to the sample loading pad of one strip is about 5 uL, 10 uL, 15 uL, 20 uL, 25 uL, 30 uL, 35 uL, 40 uL, 45 uL, or 50 uL. In embodiments, a suitable amount of semen applied to the sample loading pad of one strip is about 5 uL to about 10 uL, about 5 uL to about 15 uL, about 5 uL to about 20 uL, about 5 uL to about 25 uL, about 5 uL to about 30 uL, about 5 uL to about 25 uL, about 5 uL to about 40 uL, about 5 uL to about 45 uL, about 5 uL to about 50 uL, about 10 uL to about 15 uL, about 10 uL to about 20 uL, about 10 uL to about 25 uL, about 10 uL to about 30 uL, about 10 uL to about 25 uL, about 10 uL to about 40 uL, about 10 uL to about 45 uL, about 10 uL to about 50 uL, about 15 uL to about 20 uL, about 15 uL to about 25 uL, about 15 uL to about 30 uL, about 15 uL to about 25 uL, about 15 uL to about 40 uL, about 15 uL to about 45 uL, about 15 uL to about 50 uL, about 20 uL to about 25 uL, about 20 uL to about 30 uL, about 20 uL to about 25 uL, about 20 uL to about 40 uL, about 20 uL to about 45 uL, about 20 uL to about 50 uL, about 25 uL to about 30 uL, about 25 uL to about 25 uL, about 25 uL to about 40 uL, about 25 uL to about 45 uL, about 25 uL to about 50 uL, about 30 uL to about 25 uL, about 30 uL to about 40 uL, about 30 uL to about 45 uL, about 30 uL to about 50 uL, about 25 uL to about 40 uL, about 25 uL to about 45 uL, about 25 uL to about 50 uL, about 40 uL to about 45 uL, about 40 uL to about 50 uL, or about 45 uL to about 50 uL.

In embodiments, a suitable amount of vaginal fluid applied to the sample loading pad of one strip is about 5 uL to about 50 uL. In embodiments, a suitable amount of vaginal fluid applied to the sample loading pad of one strip is at least about 5 uL. In embodiments, a suitable amount of vaginal fluid applied to the sample loading pad of one strip is at most about 50 uL. In embodiments, a suitable amount of vaginal fluid applied to the sample loading pad of one strip is about 5 uL, 10 uL, 15 uL, 20 uL, 25 uL, 30 uL, 35 uL, 40 uL, 45 uL, or 50 uL. In embodiments, a suitable amount of vaginal fluid applied to the sample loading pad of one strip is about 5 uL to about 10 uL, about 5 uL to about 15 uL, about 5 uL to about 20 uL, about 5 uL to about 25 uL, about 5 uL to about 30 uL, about 5 uL to about 25 uL, about 5 uL to about 40 uL, about 5 uL to about 45 uL, about 5 uL to about 50 uL, about 10 uL to about 15 uL, about 10 uL to about 20 uL, about 10 uL to about 25 uL, about 10 uL to about 30 uL, about 10 uL to about 25 uL, about 10 uL to about 40 uL, about 10 uL to about 45 uL, about 10 uL to about 50 uL, about 15 uL to about 20 uL, about 15 uL to about 25 uL, about 15 uL to about 30 uL, about 15 uL to about 25 uL, about 15 uL to about 40 uL, about 15 uL to about 45 uL, about 15 uL to about 50 uL, about 20 uL to about 25 uL, about 20 uL to about 30 uL, about 20 uL to about 25 uL, about 20 uL to about 40 uL, about 20 uL to about 45 uL, about 20 uL to about 50 uL, about 25 uL to about 30 uL, about 25 uL to about 25 uL, about 25 uL to about 40 uL, about 25 uL to about 45 uL, about 25 uL to about 50 uL, about 30 uL to about 25 uL, about 30 uL to about 40 uL, about 30 uL to about 45 uL, about 30 uL to about 50 uL, about 25 uL to about 40 uL, about 25 uL to about 45 uL, about 25 uL to about 50 uL, about 40 uL to about 45 uL, about 40 uL to about 50 uL, or about 45 uL to about 50 uL.

In embodiments, a suitable amount of urine applied to the sample loading pad of one strip is about 5 uL to about 50 uL. In embodiments, a suitable amount of urine applied to the sample loading pad of one strip is at least about 5 uL. In embodiments, a suitable amount of urine applied to the sample loading pad of one strip is at most about 50 uL. In embodiments, a suitable amount of urine applied to the sample loading pad of one strip is about 5 uL, 10 uL, 15 uL, 20 uL, 25 uL, 30 uL, 35 uL, 40 uL, 45 uL, or 50 uL. In embodiments, a suitable amount of urine applied to the sample loading pad of one strip is about 5 uL to about 10 uL, about 5 uL to about 15 uL, about 5 uL to about 20 uL, about 5 uL to about 25 uL, about 5 uL to about 30 uL, about 5 uL to about 25 uL, about 5 uL to about 40 uL, about 5 uL to about 45 uL, about 5 uL to about 50 uL, about 10 uL to about 15 uL, about 10 uL to about 20 uL, about 10 uL to about 25 uL, about 10 uL to about 30 uL, about 10 uL to about 25 uL, about 10 uL to about 40 uL, about 10 uL to about 45 uL, about 10 uL to about 50 uL, about 15 uL to about 20 uL, about 15 uL to about 25 uL, about 15 uL to about 30 uL, about 15 uL to about 25 uL, about 15 uL to about 40 uL, about 15 uL to about 45 uL, about 15 uL to about 50 uL, about 20 uL to about 25 uL, about 20 uL to about 30 uL, about 20 uL to about 25 uL, about 20 uL to about 40 uL, about 20 uL to about 45 uL, about 20 uL to about 50 uL, about 25 uL to about 30 uL, about 25 uL to about 25 uL, about 25 uL to about 40 uL, about 25 uL to about 45 uL, about 25 uL to about 50 uL, about 30 uL to about 25 uL, about 30 uL to about 40 uL, about 30 uL to about 45 uL, about 30 uL to about 50 uL, about 25 uL to about 40 uL, about 25 uL to about 45 uL, about 25 uL to about 50 uL, about 40 uL to about 45 uL, about 40 uL to about 50 uL, or about 45 uL to about 50 uL.

The body fluid sample can be obtained from a patient using any appropriate method known in the art. Whole blood can be removed by finger prick with a sharp instrument, e.g., a lancet, needle, or pin. Semen, saliva, and vaginal sample material can be obtained by, e.g., swabbing. The swab can then be rinsed in buffer for loading.

In embodiments, the patient is a human, and reagents, e.g., anti-subtype antibodies, are selected accordingly as understood by one of skill in the art. In embodiments, the patient is a monkey and reagents are selected accordingly.

### Target Analyte

The presence of at least one target analyte is assayed using the compositions and methods of the invention. In embodiments, the target analyte is an analyte in a patient sample wherein its presence or absence indicates Zika virus infection status. In embodiments, the presence of at least two different target analytes are assayed using the methods and compositions described and the results used in combination to determine Zika virus infection status. As described herein, in embodiments, the presence of anti-Zika antibody and anti-Zika IgG are assayed. In embodiments, the presence of anti-Zika antibody, and any two of anti-Zika IgG, anti-Zika IgM, and anti-Zika IgA are assayed.

### Tracer Reagent

In embodiments, the tracer reagent of either of the first and second test strips is a Zika antigen selected from: recombinant Zika NS1, a fragment of recombinant Zika NS1, recombinant Zika ENV, a fragment of recombinant Zika ENV, recombinant Zika VLP, a fragment of recombinant Zika VLP, and purified Zika Virus Lysate. These tracer reagents can be obtained commercially, labeled or unlabeled, or can be produced using any appropriate known method

### Tracer Reagent Labeling

The tracer reagent, or any tracer reagent described herein, can be labeled with any appropriate labeling agent known in the art, e.g., enzymes, fluorescent tags, gold nanoparticles, quantum dots, or latex beads, using any suitable method known in the art or described in the literature. In particular embodiments, the tracer reagent is labeled with gold nanoparticles. In embodiments, the tracer reagent is labeled with gold nanoparticles as described herein in the Examples.

For example, a tracer reagent used on the first test line can comprise Zika NS1 protein (commercially available, e.g., from Ross Southern Laboratories, Utah, USA), labeled with 60 nm gold particle by mixing 50 ng particle in 10 ml in pH 7.4 phosphate buffer, centrifuging at 12,000 rpm, dissolving the pellet in pH 7.2 phosphate buffer containing 1% BSA, 1% milk, and 0.05% Tween 20, spraying the resulting solution containing to glass substrate, and vacuum drying.

### Tracer Signal Detection

In embodiments, detection of the tracer signal immobilized to a test line or control line of the first or second test strip is carried out visually, e.g., by a patient or healthcare provider. In embodiments, detection of the tracer signal is carried out by a reading device capable of identifying or both identifying and measuring the intensity of a tracer signal used in the assay. In embodiments, the intensity of the test signal present at a test or control line indicates the level of target analyte in the patient sample.

### Immobilized Detection Reagent

The detection reagents at the test or control lines described herein can be immobilized to a test strip at the desired test line by any appropriate means known in the art for immobilizing a protein to a lateral flow test strip. Methods for immobilizing proteins to nitrocellulose test strips are described, e.g., by Holstein, et al., 2016, incorporated herein by reference. In embodiments, the immobilized detection reagent at a test line of a test strip of the invention is present at a uniform density or concentration on the test line. In these embodiments, a simple readout of the presence or absence of the test signal (+ or −) can be made visually or by machine. In embodiments, the detection reagent is immobilized at a density or concentration for suitable for a quantitation of the test signal. In certain embodiments, the density or concentration of the detection reagent immobilized to the test line results in a gradient of the reagent, e.g., progressing from less concentrated to more concentrated in the direction of flow, such that the position of the test signal on the test line provides a quantitative readout or relative comparison of analyte concentration, e.g., when aligned with a standard. Quantitative formats for lateral flow assays are discussed, e.g., by Sajid, et al., 2014, incorporated herein by reference.

### Zika Antigen

As described herein, a detection reagent, e.g., as used at the first test line, can comprise a Zika antigen selected from: recombinant Zika NS1, a fragment of recombinant Zika NS1, recombinant Zika ENV, a fragment of recombinant Zika ENV, recombinant Zika VLP, a fragment of recombinant Zika VLP, and purified Zika Virus Lysate. Useful reagents are commercially available, from, e.g., Fitzgerald, Acton, Mass., and BioFront Technologies, Tallahassee, Fla.

### Anti-Human Subtype Antibody

As described herein, detection reagents, e.g., as used at the second and third test lines, can comprise anti-human subtype antibodies, e.g., mAbs, selected from anti-IgG, anti-IgM, and anti-IgA. Anti-human subtype antibodies, and other test strip reagents, are commercially available, labeled or unlabeled. For example, anti-human IgG, anti-human IgM and Zika virus extract or Zika virogenetic protein useful in the methods and compositions of the present invention can be obtained from JAJ International, Inc., San Diego, Calif.

### Anti-Zika Antigen Antibody

As described herein, detection reagents, e.g., as used at the first and second control lines, can comprise anti-Zika antigen antibodies selected to bind to the tracer antigen.

In embodiments, a control line detection reagent can be selected to bind to an additional tracer reagent that has been mixed with the tracer reagent that binds to the target analyte.

### Assays

An assay of the present invention comprises at least a first and second test strip in any configuration as described herein. In embodiments, the first test strip comprises a sandwich assay that tests for a target analyte, e.g., an anti-Zika antibody. In embodiments, the second test strip comprises an anti-Zika antibody subtype assay, wherein the antibody subtype tested is, e.g., IgG, IgM or IgA, or a combination thereof. In embodiments, the second test strip comprises two different antibody subtype assays, wherein the two antibody subtypes tested are, e.g., IgG, IgM or IgA. In embodiments, the assay comprises two antibody subtype test strips, wherein each test strip comprises a different antibody subtype assay.

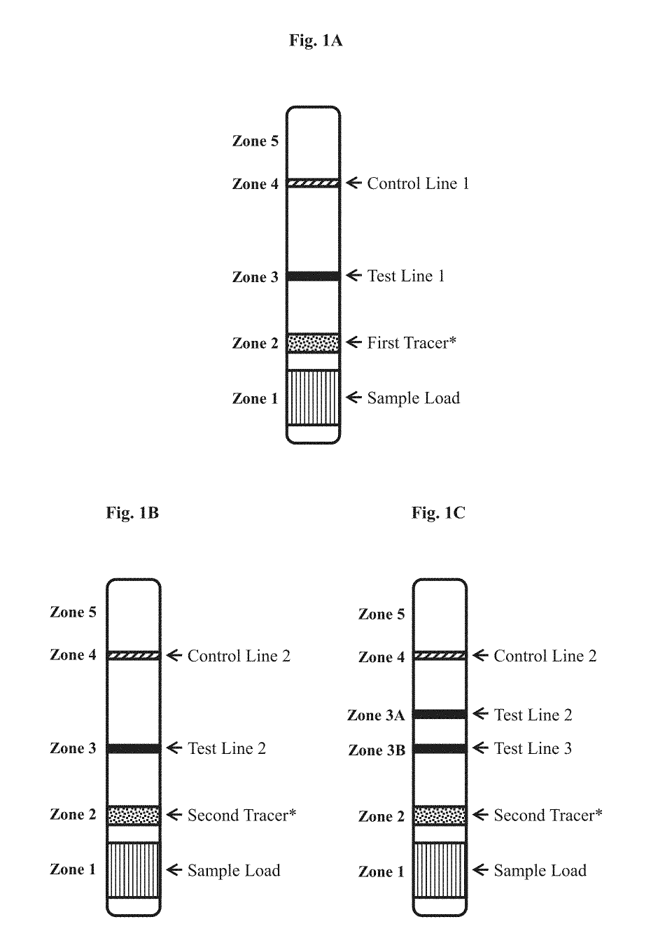

In embodiments, the assay comprises two test strips, wherein the first test strip comprises an anti-Zika antibody assay as described by FIG. 1A, and the second test strip comprises an anti-Zika IgG subtype antibody assay as described by FIG. 1B.

In specific embodiments, the assay comprises first and second test strips, wherein the first test strip comprises an anti-Zika NS1 antibody assay as described by FIG. 1A, as follows: the first tracer reagent is labeled Zika NS1; the immobilized detection reagent at the first test line is Zika NS1; and the immobilized control line reagent at the first control line is an anti-Zika NS1 antibody; and wherein the second test strip comprises an anti-Zika IgG subtype antibody assay as described by FIG. 1B, as follows: the second conjugated tracer reagent is labeled Zika NS1; the immobilized detection reagent at the second test line is an anti-human IgG antibody; and the immobilized control line reagent at the second control line is an anti-Zika NS1 antibody.

In embodiments, the assay comprises two test strips, wherein the first test strip comprises an anti-Zika antibody assay as described by FIG. 1A, and the second test strip comprises an anti-Zika IgM subtype antibody assay as described by FIG. 1B.

In specific embodiments, the assay comprises first and second test strips, wherein the first test strip comprises an anti-Zika NS1 antibody assay as described by FIG. 1A, as follows: the conjugated tracer reagent is labeled Zika NS1; the immobilized detection reagent at the first test line is Zika NS1; and the immobilized control line reagent at the first control line is an anti-Zika NS1 antibody; and wherein the second test strip comprises an anti-Zika IgM subtype antibody assay as described by FIG. 1B, as follows: the conjugated tracer reagent is labeled Zika NS1; the immobilized detection reagent at the second test line is an anti-human IgM antibody; and the immobilized control line reagent at the second control line is an anti-Zika NS1 antibody.

In embodiments, the assay comprises two test strips, wherein the first test strip comprises an anti-Zika antibody assay as described by FIG. 1A, and the second test strip comprises an anti-Zika IgG subtype antibody assay and an anti-Zika IgM subtype antibody assay as described by FIG. 1C.

In specific embodiments, the assay comprises first and second test strips, wherein the first test strip comprises an anti-Zika NS1 antibody assay as described by FIG. 1A, as follows: the conjugated tracer reagent is labeled Zika NS1; the immobilized detection reagent at the first test line is Zika NS1; and the immobilized control line reagent at the first control line is an anti-Zika NS1 antibody; and wherein the second test strip comprises an anti-Zika IgG subtype antibody assay and an anti-Zika IgM subtype antibody assay as described by FIG. 1C, as follows: the conjugated tracer reagent is labeled Zika NS1; the immobilized detection reagent at the second test line is an anti-human IgG antibody; the immobilized detection reagent at the third test line is an anti-human IgM antibody; and the immobilized control line reagent is an anti-Zika NS1 antibody.

In embodiments, the assay comprises three test strips, wherein the first test strip comprises an anti-Zika antibody assay as described by FIG. 1A, the second test strip comprises an anti-Zika IgG subtype antibody assay as described by FIG. 1B, and the third test strip comprises an anti-Zika IgM subtype antibody assay also as described by FIG. 1B.

In specific embodiments, the assay comprises first, second and third test strips, wherein the first test strip comprises an anti-Zika NS1 antibody assay as described by FIG. 1A, as follows: the first tracer reagent is labeled Zika NS1; the immobilized detection reagent at the first test line is Zika NS1; and the immobilized control line reagent at the first control line is an anti-Zika NS1 antibody; wherein the second test strip comprises an anti-Zika IgG subtype antibody assay as described by FIG. 1B, as follows: the second conjugated tracer reagent is labeled Zika NS1; the immobilized detection reagent at the second test line is an anti-human IgG antibody; and the immobilized control line reagent at the second control line is an anti-Zika NS1 antibody; and wherein the third test strip comprises an anti-Zika IgM subtype antibody assay as described by FIG. 1B, as follows: the conjugated tracer reagent is labeled Zika NS1; the immobilized detection reagent at the second test line is an anti-human IgM antibody; and the immobilized control line reagent at the second control line is an anti-Zika NS1 antibody.

### Assay Devices

An assay device of the present invention is a device that facilitates the operation of any immunchromatographic lateral flow test strip assay described herein. In embodiments, the assay device contains the test strips in a single or multiple housings. The housing provides, e.g., ease of use and/or protection of the strips and reagents. The device configuration or format can be any convenient format known in the art, e.g., the test strips may be housed in parallel in a cassette. In embodiments, the housing comprises any suitable material, in particular, a material that does not react with the assay components or the patient sample to be analyzed, as can be selected by one of skill in the art. In embodiments, the housing is a plastic cassette that encases one or more test strips and has windows to allow sample loading and reading of the test results.

In embodiments, an assay or assay device composition of the present invention is stable for at least about 1 month, 3 months, 6 months, 12 months, 18 months, or 24 months when stored at about 4 degrees C. to about 30 degrees C. In embodiments, a assay or device composition of the present invention is stable for about 1 to about 6, about 1 to about 12, about 1 to about 18, or about 1 to about 24 months when stored at about 4 degrees C. to about 30 degrees C.

In embodiments, an assay or assay device composition of the present invention is stable for at least about 1 month, 3 months, 6 months, 12 months, 18 months, or 24 months when stored at room temperature. In embodiments, a assay or device composition of the present invention is stable for about 1 to about 6, about 1 to about 12, about 1 to about 18, or about 1 to about 24 months when stored at room temperature.

### Operation

Operation of the assay, contained in or independent of one or more housing components, comprises application of the test sample to the sample pad of each test strip as described herein. Application can be carried out by use of any appropriate instrument as known in the art, e.g., a dropper or pipet, to apply an appropriate sample volume. In embodiments, the assay result is ready to read in about 5 to about 20 minutes. In embodiments, the assay result is ready to read in about 5, 6, 7, 8, 9, 10,11, 12, 13, 14, 15, 16, 17, 18, 19, 20, 21, 22, 23, 24, 25, or 30 minutes. In embodiments, the assay result is ready to read in about 5 minutes to about 30 minutes. In embodiments, the assay result is ready to read in about 5 minutes to about 30 minutes. In embodiments, the assay result is ready to read in at most about 5, 10, 15, 20, 25 or 30 minutes. In embodiments, the assay result is ready to read in about 5 minutes to about 10 minutes, about 5 minutes to about 15 minutes, about 5 minutes to about 16 minutes, about 5 minutes to about 17 minutes, about 5 minutes to about 18 minutes, about 5 minutes to about 19 minutes, about 5 minutes to about 20 minutes, about 5 minutes to about 21 minutes, about 5 minutes to about 22 minutes, about 5 minutes to about 25 minutes, about 5 minutes to about 30 minutes, about 10 minutes to about 15 minutes, about 10 minutes to about 16 minutes, about 10 minutes to about 17 minutes, about 10 minutes to about 18 minutes, about 10 minutes to about 19 minutes, about 10 minutes to about 20 minutes, about 10 minutes to about 21 minutes, about 10 minutes to about 22 minutes, about 10 minutes to about 25 minutes, about 10 minutes to about 30 minutes, about 15 minutes to about 16 minutes, about 15 minutes to about 17 minutes, about 15 minutes to about 18 minutes, about 15 minutes to about 19 minutes, about 15 minutes to about 20 minutes, about 15 minutes to about 21 minutes, about 15 minutes to about 22 minutes, about 15 minutes to about 25 minutes, about 15 minutes to about 30 minutes, about 16 minutes to about 17 minutes, about 16 minutes to about 18 minutes, about 16 minutes to about 19 minutes, about 16 minutes to about 20 minutes, about 16 minutes to about 21 minutes, about 16 minutes to about 22 minutes, about 16 minutes to about 25 minutes, about 16 minutes to about 30 minutes, about 17 minutes to about 18 minutes, about 17 minutes to about 19 minutes, about 17 minutes to about 20 minutes, about 17 minutes to about 21 minutes, about 17 minutes to about 22 minutes, about 17 minutes to about 25 minutes, about 17 minutes to about 30 minutes, about 18 minutes to about 19 minutes, about 18 minutes to about 20 minutes, about 18 minutes to about 21 minutes, about 18 minutes to about 22 minutes, about 18 minutes to about 25 minutes, about 18 minutes to about 30 minutes, about 19 minutes to about 20 minutes, about 19 minutes to about 21 minutes, about 19 minutes to about 22 minutes, about 19 minutes to about 25 minutes, about 19 minutes to about 30 minutes, about 20 minutes to about 21 minutes, about 20 minutes to about 22 minutes, about 20 minutes to about 25 minutes, about 20 minutes to about 30 minutes, about 21 minutes to about 22 minutes, about 21 minutes to about 25 minutes, about 21 minutes to about 30 minutes, about 22 minutes to about 25 minutes, about 22 minutes to about 30 minutes, or about 25 minutes to about 30 minutes.

### Assay Specificity

In embodiments, the compositions and methods of invention provide high assay specificity resulting in a low rate of false positive results. In embodiments, high assay specificity is represented by low cross-reactivity with other infections. Cross-reactivity can result in determination of a positive (early, late, or intermediate) Zika virus infection status upon analysis of a sample that contains antibody to a different infectious agent but does not contain anti-Zika antibody or does not a contain significant level of anti-Zika antibody. In embodiments, the different infectious agent is a related virus, e.g., dengue, West Nile, or yellow fever virus.

In other embodiments, a false positive result arises in a sample from a patient not infected with Zika virus or a different infectious agent. In embodiments, high assay specificity is represented by a low rate of false positives.

In embodiments, the presence or absence of cross-reactivity is confirmed using any appropriate method as known to one of skill in the art or described in the literature, including, but not limited to antibody testing, e.g., ELISA, or nucleic acid analysis, e.g, using PCR methods.

In embodiments, a low or acceptable level of false positives, resulting from, e.g., cross-reactivity, is represented by an occurrence of cross-reactivity or false positives, at less than 20%. In embodiments, an acceptable rate or very low rate of cross-reactivity or false positives is represented by an occurrence of cross-reactivity or false positive results, at less than 20%, less than 19%, less than 18%, less than 17%, less than 16%, less than 15%, less than 14%, less than 13%, less than 12%, less than 11%, less than 10%, less than 9%, less than 8%, less than 7.7%, less than 7%, less than 6.5%, less than 6%, less than 5%, less than 4%, less than 3%, less than 2%, less than 1%, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, 2% or less, 1% or less, or 0%.

In embodiments, an acceptable level of cross-reactivity, e.g., low cross-reactivity, or low false positives, is represented by an absence of cross-reactivity or false positive results, of 80% or greater. In embodiments, an acceptable level of cross-reactivity, e.g., low cross-reactivity, or low false positives, is represented by an absence of cross-reactivity or false positive results of about 80% or greater, about 81% or greater, about 82% or greater, about 83% or greater, about 84% or greater, about 85% or greater, about 86% or greater, about 87% or greater, about 88% or greater, about 89% or greater, about 90% or greater, about 91% or greater, about 92% or greater, about 92.3% or greater, about 93% or greater, about 93.5% or greater, about 94% or greater, about 95% or greater, about 96% or greater, about 97% or greater, about 98% or greater, about 99% or greater, or 100%.

In specific embodiments, the occurrence of cross-reactivity with dengue virus resulting in a false positive when using an assay of the present invention to determine early or intermediate Zika infection status is less than about 10%, less than about 9%, less than about 8%, less than about 7.7%, less than about 7%, less than about 6%, less than about 5%, less than about 4%, less than about 3%, less than about 2%, less than about 1%, 10% or less, 9% or less, 8% or less, 7% or less, 6% or less, 5% or less, 4% or less, 3% or less, 2% or less, 1% or less, or 0%.

In embodiments, the specificity of an assay of the present invention is represented by a high correlation of the specificity test results with the results for the same samples obtained in a second assay, e.g., ELISA or PCR. In embodiments, the correlation of the test results with the results for the same samples obtained in a second assay is 90% or greater. In embodiments, the correlation of the test results with the results for the same samples obtained in a second assay is at least about 90%, at least about 91%, at least about 92%, at least about 93%, at least about 94%, at least about 95%, at least about 96%, at least about 97%, at least about 98%, at least about 99%, or 100%. In embodiments, the correlation of the test results with the results for the same samples obtained in a second assay is about 80% to 100%. In embodiments, the correlation of the test results with the results for the same samples obtained in a second assay is about 80% to about 100%. In embodiments, the correlation of the test results with the results for the same samples obtained in a second assay is at least about 80%. In embodiments, the correlation of the test results with the results for the same samples obtained in a second assay is about 80% to about 85%, about 80% to about 90%, about 80% to about 92%, about 80% to about 93%, about 80% to about 94%, about 80% to about 95%, about 80% to about 96%, about 80% to about 97%, about 80% to about 98%, about 80% to about 99%, about 80% to about 100%, about 85% to about 90%, about 85% to about 92%, about 85% to about 93%, about 85% to about 94%, about 85% to about 95%, about 85% to about 96%, about 85% to about 97%, about 85% to about 98%, about 85% to about 99%, about 85% to about 100%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%.

### Assay Sensitivity

In embodiments, the compositions and methods of invention provide high assay sensitivity. In embodiments, assay sensitivity is represented by a low or acceptable rate of false negative results. In embodiments, a low or acceptable rate of false negative results is represented by the occurrence of false negative results at less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, 2% or less, 1% or less, or 0%.

In embodiments, a low or acceptable level of false negative results, is represented by an absence of false negative results of about 80% or greater, about 81% or greater, about 82% or greater, about 83% or greater, about 84% or greater, about 85% or greater, about 86% or greater, about 87% or greater, about 88% or greater, about 89% or greater, about 90% or greater, about 91% or greater, about 92% or greater, about 93% or greater, about 94% or greater, about 95% or greater, about 96% or greater, about 97% or greater, about 98% or greater, about 99% or greater, or 100%.

In specific embodiments, the occurrence of false negative results when using an assay of the present invention to determine early Zika infection status is 10% or less, 9% or less, 8% or less, 7% or less, 6% or less, 5% or less, 4% or less, 3% or less, 2% or less, 1% or less, less than 10%, less than 9%, less than 8%, less than 7%, less than 6%, less than 5%, less than 4%, less than 3%, less than 2%, less than 1%, 10% or less, 9% or less, 8% or less, 7% or less, 6% or less, 5% or less, 4% or less, 3% or less, 2% or less, 1% or less, or 0%.

In embodiments, the sensitivity of an assay of the present invention is represented by a high correlation of the sensitivity test results with the results for the same samples obtained in a second assay, e.g., ELISA or PCR. In embodiments, the correlation of the test results with the results for the same samples obtained in a second assay is 90% or greater. In embodiments, the correlation of the test results with the results for the same samples obtained in a second assay is at least about 90%, at least about 91%, at least about 92%, at least about 93%, at least about 94%, at least about 95%, at least about 96%, at least about 97%, at least about 98%, at least about 99%, or 100%. In embodiments, the correlation of the test results with the results for the same samples obtained in a second assay is about 80% to 100%. In embodiments, the correlation of the test results with the results for the same samples obtained in a second assay is about 80% to about 100%. In embodiments, the correlation of the test results with the results for the same samples obtained in a second assay is at least about 80%. In embodiments, the correlation of the test results with the results for the same samples obtained in a second assay is about 80% to about 85%, about 80% to about 90%, about 80% to about 92%, about 80% to about 93%, about 80% to about 94%, about 80% to about 95%, about 80% to about 96%, about 80% to about 97%, about 80% to about 98%, about 80% to about 99%, about 80% to about 100%, about 85% to about 90%, about 85% to about 92%, about 85% to about 93%, about 85% to about 94%, about 85% to about 95%, about 85% to about 96%, about 85% to about 97%, about 85% to about 98%, about 85% to about 99%, about 85% to about 100%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%.

### Confirmatory Assays

In embodiments, the methods of the invention further include confirming the infection status determined using another assay. In embodiments, the confirmatory assay is an antibody assays or a nucleic acid assay. In embodiments, the confirmatory assay is ELISA, PCR, or a combination of assays, e.g., Multiplex Luminex Serology (Pasteur Test).

### Assay Kits

An assay kit of the invention comprises a two-strip immunochromatographic lateral flow assay as described herein, in a single housing e.g., in a cassette holding the two strips, or in separate housings. The assay kit can also include an amount of a chase buffer, e.g., PBS, sufficient to enable proper flow of the tracer reagent on each of the first and second test strips to the control line on each test strip, and to enable the flow of the complex comprising the tracer reagent on each of the first and second test strips, to each respective test line on each test strip. For example, where about 40 uL of chase buffer is sufficient to properly operate each strip, the kit may include at least about 50 uL to about 100 uL of chase buffer per strip. Additional kit components can include, e.g., an instrument for sample collection, e.g., a sharp instrument for drawing blood, or a swab for collecting saliva, urine, semen, or vaginal fluid, and an instrument for applying the sample to the sample pad, e.g., a dropper.

The assay kit can further include instructions for use, which can comprise a description of test pattern interpretation, and recommendations for patient action based on the result obtained. In embodiments, the patient is encouraged to seek a confirmatory test should the rapid test of the invention indicate early or intermediate Zika virus infection. In embodiments, contact information for a suitable test facility is provided.

In further embodiments, the instructions for use include a cautionary warning based on the result interpretation. For example, the CDC recommends that women who have been infected or exposed to ZIKV wait at least 8 weeks from symptom onset or last exposure before attempting conception (Petersen, et. al., “Update: interim guidance for health care providers caring for women of reproductive age with possible Zika virus exposure—United States, 2016,” MMWR Morbidity and Mortality Weekly Report 2016; 65:315-322, incorporated by reference herein). Given this recommendation, a suggestion can be made to practice safe sex or avoid conception upon observation of an early or intermediate infection status. In embodiments, a mobile phone application is made available to the user, so that test results may be provided to a practitioner and/or epidemiologist.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1. Construction of Immunochromatographic Lateral Flow Anti-Zika IgG Assay Devices

### Experiment I and II Devices

For each of Experiments I and II, described in Example 2, two-strip immunochromatographic lateral flow anti-Zika IgG assay device was constructed.

### First Test Strip—Antibody Sandwich Assay

The first test strip, formatted as generally set forth in FIG. 1A, was made for detecting anti-Zika antigen in a test sample.

Conjugate Pad (Zone 2): Gold-labeled Zika NS1 protein was sprayed and dried onto a glass fiber conjugate pad of the first test strip for use as the tracer reagent. The Zika NS1 protein (Ross Southern Laboratories, Utah) was labeled with 60 nm gold particles prepared by adding 50 ng particles in 10 ml pH 7.4 phosphate buffer, mixing well, followed by centrifugation at 12,000 rpm. The resulting pellet was dissolved in phosphate buffer pH 7.2 containing 1% BSA, 1% Milk, and 0.05% Tween 20. This solution was sprayed onto the conjugate pad and vacuum dried.

Test Line 1 (Zone 3): Zika NS1 protein (from Ross Southern Laboratories, Utah) was immobilized on nitrocellulose membrane (Sartorius, Bohemia, N.Y.) at the first test line. 1.2 mg/ml antigen protein in phosphate buffer pH 7.4, was dispensed.

Control Line 1 (Zone 4): Mouse antibody specific for the Zika NS1 tracer was immobilized on the nitrocellulose membrane. A solution of 0.5 mg/ml of anti-Zika-NS1 mouse antibody (Fitzgerald Industries International, MA, USA) in phosphate buffer pH 7.4 was used.

### Second Test Strip—Antibody Subtype Assay (IgG)

The second test strip, formatted as generally set forth in FIG. 1B, was made for detecting an anti-Zika antibody IgG subtype in the sample. The conjugate pad was made as described for the first test strip, also including nonspecific mouse antibody.

Test Line 2 (Zone 3): An anti-human IgG Mouse monoclonal antibody (Artron Laboratories, Inc., Burnaby, BC, Canada) was immobilized on the nitrocellulose membrane. 2.0 mg/ml antibody in phosphate buffer pH 7.4, was dispensed.

Control Line 2 (Zone 4): Goat anti-mouse antibody was immobilized on the nitrocellulose membrane. A solution of 2.0 mg/ml antibody (Artron Laboratories, Inc., Burnaby, BC, Canada) in phosphate buffer pH 7.4 was used.

The glass fiber conjugate pad and nitrocellulose membrane absorbent paper for each test strip were assembled on an adhesive card (backing), with overlap of around 1 mm for each pad and membrane. Strips were then cut to an appropriate size for insertion in a plastic cassette housing.

### Example 2. Anti-Zika IgG Sandwich Assay Serum Sample Analysis—Preliminary Testing

In two separate experiments (Experiments I and II), described below, test strips constructed as described in Example 1 were used to test serum samples determined to be anti-Zika IgM positive or anti-Zika IgM negative by an ELISA (Euroimmun US, New Jersey). The presence of IgG was not tested by the ELISA. In each experiments, 20 uL of serum was loaded into sample well (sample pad, Zone 1) of the first test strip in the device, followed by 40 uL of chase buffer (PBS). 5 uL serum was loaded into the sample well of the second test strip in the device, followed by 60 uL of chase buffer (PBS). Test results for both strips were read at 20 minutes.

### Experiment I Results

As shown in Table 1 all serum samples that tested as negative for anti-Zika IgM by ELISA also tested as anti-Zika IgM negative using the two-strip immunochromatographic lateral flow assay.

TABLE 1
IgM Negative Serum Sample Test - Experiment I

| | #1 | #2 | #3 | #4 | #5 | #6 | #7 | #8 | #9 | #10 | #11 | #12 | #13 | #14 |
|---|---|---|---|---|---|---|---|---|---|---|---|---|---|---|
| Test | — | — | — | — | — | — | — | — | — | — | — | — | — | — |
| Line 1 | | | | | | | | | | | | | | |
| Test | — | — | — | — | — | — | — | — | — | — | — | — | — | — |
| Line 2 | | | | | | | | | | | | | | |

Table 2 below shows the results obtained for the serum samples determined to be IgM positive by ELISA.

TABLE 2
IgM Positive Serum Sample Test - Experiment I

| | #1 | #2 | #3 | #4 | #5 | #6 | #7 | #8 | #9 |
|---|---|---|---|---|---|---|---|---|---|
| Test | + | + | + | − | + | + | − | + | + |
| Line 1 | | | | | | | | | |
| Test | − | − | − | − | + | − | − | − | + |
| Line 2 | | | | | | | | | |

### Experiment II Results

As shown in Table 3 all serum samples that tested as negative for anti-Zika IgM by ELISA also tested as IgM negative using the two-strip immunochromatographic lateral flow assay.

TABLE 3
IgM Negative Serum Sample Test - Experiment II

| | #1 | #2 | #3 | #4 | #5 | #6 | #7 | #8 | #9 | #10 | #11 | #12 | #13 | #14 |
|---|---|---|---|---|---|---|---|---|---|---|---|---|---|---|
| Test | — | — | — | — | — | — | — | — | — | — | — | — | — | — |
| Line 1 | | | | | | | | | | | | | | |
| Test | — | — | — | — | — | — | — | — | — | — | — | — | — | — |
| Line 2 | | | | | | | | | | | | | | |

Table 4 shows the results obtained for the serum samples determined to be IgM positive by ELISA.

TABLE 4
IgM Positive Serum Sample Test - Experiment II

| | #1 | #2 | #3 | #4 | #5 | #6 | #7 | #8 | #9 |
|---|---|---|---|---|---|---|---|---|---|
| Test | + | − | + | − | + | + | − | + | − |
| Line 1 | | | | | | | | | |
| Test | − | − | + | − | + | − | − | − | − |
| Line 2 | | | | | | | | | |

### Example 3. Anti-Zika IgG Sandwich Assay Serum Sample Analysis

An experiment testing 49 serum samples determined to be anti-Zika IgM positive by ELISA (Euroimmun US, New Jersey), 10 normal serum samples, and five dengue positive samples was conducted using a two-strip immunochromatographic lateral flow anti-Zika IgG assay. The first test strip used, as tracer, recombinant NS1 protein, with recombinant NS1 protein immobilized to the first test line. The second test strip also used, as tracer, recombinant NS1 protein, and had immobilized mouse anti-human IgG mAb at the second test line.

A summary of the results obtained at 20 minutes after sample load is shown in Table 5. Table 5 shows that of the 49 samples determined Zika positive by ELISA, 45/49 (92%) also were determined to be positive by the two-strip anti-Zika IgG sandwich assay. (+++=strong positive signal; ++=clear positive signal; +=faint positive signal; Equivocal=fewer than 4/4 observers called result+; Negative=all 4/4 observers called result negative.)

TABLE 5
Summary of 64 Serum Sample Test Using Anti-Zika IgG Sandwich Assay

| Serum | No. | Total Ab by Sandwich Assay | | | | | | IgG |
|---|---|---|---|---|---|---|---|---|
| Sample | Tested | +++ | ++ | + | Total Pos | Equivocal | Negative | Negative |
| Zika Positive | 49* | 3 | 20 | 22 | 45 | 4 | 0 | 10** |
| Normal | 10 | 0 | 0 | 0 | 0 | 0 | 10 | 10 |
| Dengue | 5 | 0 | 0 | 0 | 0 | 1 | 4 | 5 |
| Positive | | | | | | | | |

*One sample could not be evaluated because control was weak.
**Of the 10 IgG-negative samples, 7 were confirmed to be positive and 3 equivocal for IgM to Zika.

### Example 4. Anti-Zika IgG and Anti-IgM Sandwich Assay Serum Sample Analysis

An experiment testing 51 blood samples determined to be Zika positive by Multiplex Luminex Serology, was carried out using a two-strip immunochromatographic lateral flow anti-Zika IgG and IgM assay (by the Central National Laboratory, Brazil). The first test strip of the assay used, as tracer, recombinant NS1 protein, with recombinant NS1 protein immobilized to the first test line. The second test strip also used, as tracer, recombinant NS1 protein, and had immobilized mouse anti-human IgG mAb at the second test line and immobilized mouse anti-human IgM mAb at the third test line.

Table 6 shows the results obtained using the two-strip immunochromatographic lateral flow anti-Zika IgG and IgM assay. A high correlation of the positive serology results was observed, with three samples showing potential cross-reactivity with another virus (3, 48 and 39).

TABLE 6
Results of 51 Serum Sample Test Using
Anti-Zika IgG and IgM Sandwich Assay

| Blood | Ab | IgG | IgM |
|---|---|---|---|
| Specimen | (Test Line 1) | (Test Line 2) | (Test Line 3) |
| 1 | + | + | +++ |
| 2 | +/− | +/− | ++ |
| 3 | − | +/− | ++ |
| 4 | + | + | +++ |
| 5 | + | + | + |
| 6 | + | − | + |
| 7 | ++ | +/− | ++ |
| 8 | ++ | +/− | + |
| 9 | +/− | − | ++ |
| 10 | ++ | +/− | + |
| 11 | + | + | +++ |
| 12 | + | +/− | ++ |
| 13 | ++ | + | + |
| 14 | +/− | − | +++ |
| 15 | + | +/− | + |
| 16 | + | ++ | ++ |
| 17 | + | +/− | ++ |
| 18 | ++ | ++ | + |
| 19 | ++ | +/− | ++ |
| 20 | ++ | + | + |
| 21 | + | + | ++ |
| 22 | + | − | +++ |
| 23 | + | +/− | − |
| 24 | ++ | +/− | + |
| 25 | + | −/+ | +++ |
| 26 | + | + | ++ |
| 27 | + | +/− | +++ |
| 28 | +/− | +/− | +++ |
| 29 | + | + | + |
| 30 | ++ | +/− | ++ |
| 31 | ++ | − | +++ |
| 32 | + | + | + |
| 33 | + | + | + |
| 34 | ++ | +/− | +++ |
| 35 | ++ | + | + |
| 36 | + | + | + |
| 37 | ++ | + | + |
| 38 | ++ | + | +++ |
| 39 | − | − | + |
| 40 | +/− | +/− | +++ |
| 41 | ++ | + | +++ |
| 42 | + | +/− | ++ |
| 43 | + | +/− | + |
| 44 | + | + | + |
| 45 | +/− | +/− | ++ |
| 46 | + | + | + |
| 47 | ++ | ++ | ++ |
| 48 | − | − | ++ |
| 49 | + | ++ | + |
| 50 | ++ | + | ++ |
| 51 | ++ | +/− | ++ |

1. A method for rapid assay of a human patient sample to determine Zika virus infection status in the patient, comprising:
comparing a test signal produced at a first test line on a first lateral flow test strip exposed to the patient sample, and a test signal produced at a second test line on a second lateral flow test strip exposed to the patient sample, wherein:
a. the presence of the test signal produced at the first test line indicates the presence of an anti-Zika antibody in the patient sample; and
b. the presence of the test signal produced at the second test line indicates the presence of one of an anti-Zika-IgG or an anti-Zika IgM in the patient sample;
wherein the test signal at each of the first and second test lines is produced in five to twenty minutes following application of the patient sample to a sample loading pad on the corresponding lateral flow test strip, and wherein when the test signal produced at the second test line indicates the presence of anti-Zika IgG in the patient sample, the Zika virus infection status determined is selected from: no Zika virus infection and early Zika virus infection, and wherein when the test signal produced at the second test line indicates the presence of anti-Zika IgM in the patient sample, the Zika virus infection status determined is selected from: no Zika virus infection and late Zika virus infection.

2. The method of claim 1, wherein the test signal at the first test line that indicates the presence of the anti-Zika antibody in the patient sample is produced by binding of the anti-Zika antibody to a first Zika tracer antigen and to a first immobilized Zika detection antigen on the first test line, whereby a complex comprising the first Zika tracer antigen—anti-Zika antibody—first immobilized Zika detection antigen is formed and produces a test signal at the first test line.

3. The method of claim 2, wherein the test signal produced at the second test line:
i. indicates the presence in the patient sample of an anti-Zika IgG, wherein the test signal at the second test line is produced by binding of the anti-Zika IgG to a second Zika tracer antigen on the second lateral flow test strip and to an immobilized anti-human-IgG antibody on the second test line, whereby a complex comprising the second Zika tracer antigen—anti-Zika IgG—immobilized anti-human-IgG antibody is formed, thereby producing a test signal at the second test line; or
ii. indicates the presence in the patient sample of an anti-Zika IgM, wherein the test signal at the second test line is produced by binding of the anti-Zika IgM to a second Zika tracer antigen on the second lateral flow test strip and to an immobilized anti-human-IgM antibody on the second test line, whereby a complex comprising the second Zika tracer antigen—anti-Zika IgM—immobilized anti-human-IgM antibody is formed, thereby producing a test signal at the second test line.

4. The method of claim 3(i), wherein the second lateral flow test strip further comprises a third test line that is separate from the second test line, wherein the presence of a test signal produced at the third test line indicates the presence of an anti-Zika IgM in the patient sample, wherein the test signal at the third test line is produced by binding of the anti-Zika IgM to the second Zika tracer antigen on the second lateral flow test strip and to an immobilized anti-human-IgM antibody on the third test line, whereby a complex comprising the second Zika tracer antigen—anti-Zika IgM—immobilized anti-human-IgM antibody is formed thereby producing a test signal at the third test line, and wherein the Zika virus infection status determined is selected from: no Zika virus infection, early Zika virus infection, intermediate Zika virus infection, and late Zika virus infection.

5. The method of claim 3(i), wherein determination of early Zika virus infection is made based on the presence of an anti-Zika antibody test signal at the first test line and the absence of an anti-Zika IgG test signal at the second test line.

6. The method of claim 3(ii), wherein determination of late Zika virus infection is made based on the presence of an anti-Zika antibody test signal at the first test line and the absence of an anti-Zika IgM test signal at the second test line.

7. The method of claim 4, wherein determination of early Zika virus infection is made based on the presence of an anti-Zika antibody test signal at the first test line, the absence of an anti-Zika IgG test signal at the second test line, and the presence of an anti-Zika IgM test signal at the third test line.

8. The method of claim 4, wherein determination of intermediate Zika virus infection is made based on the presence of an anti-Zika antibody test signal at the first test line, the presence of an anti-Zika IgG test signal at the second test line, and the presence of an anti-Zika IgM test signal at the third test line.

9. The method of claim 4, wherein determination of late Zika virus infection is made based on the presence of an anti-Zika antibody test signal at the first test line, the presence of an anti-Zika IgG test signal at the second test line, and the absence of an anti-Zika IgM test signal at the third test line.

10. The method of claim 3, wherein the determination of no Zika virus infection is based on the absence of an antibody test signal at the first test line.

11. The method of claim 1, wherein the determination of early Zika virus infection, intermediate Zika virus infection, or late Zika virus infection does not represent infection with Dengue, Chikungunya, yellow fever, malaria, or West Nile virus.

12. An assay for determining Zika virus infection status of a patient by analyzing a sample obtained from the patient, comprising:
a. a first lateral flow test strip comprising, in the direction of flow, a sample application pad, a conjugate pad comprising a first Zika tracer antigen, a first test line comprising a first immobilized Zika detection antigen, and a first control line, wherein in the presence of an anti-Zika antibody in the patient sample a complex comprising the first Zika tracer antigen—anti-Zika antibody—first immobilized Zika detection antigen is formed and produces a test signal at the first test line;
b. a second lateral flow test strip comprising, in the direction of flow, a sample application pad, a conjugate pad comprising a second Zika tracer antigen, a second test line comprising an immobilized anti-human-antibody specific for one of a human IgG and a human IgM, and a second control line, wherein in the presence of an anti-Zika IgG or IgM, respectively, in the patient sample, a complex comprising the second Zika tracer antigen—anti-Zika antibody—immobilized antibody specific for the human IgG or IgM is formed and produces a test signal at the second test line;
wherein the first and second Zika tracer antigens are the same or different, and wherein when the immobilized anti-human antibody is specific for human IgG the Zika virus infection status determined is selected from: no Zika virus infection and early Zika virus infection, and wherein when the immobilized anti-human antibody is specific for human IgM the Zika virus infection status determined is selected from: no Zika virus infection and late Zika virus infection.

13. The assay of claim 12, wherein the second lateral flow test strip further comprises, beyond the conjugate pad in the direction of sample flow, and separate from the second test line, a third test line comprising an immobilized anti-human-IgM antibody, wherein in the presence of an anti-Zika IgM in the patient sample a complex comprising the second Zika tracer antigen—anti-Zika IgM—immobilized anti-human-IgM antibody is formed and produces a test signal at the third test line, wherein the third Zika tracer antigen is the same or different from the first and second Zika tracer antigens, and wherein the Zika virus infection status determined is selected from: no Zika virus infection, early Zika virus infection, intermediate Zika virus infection, and late Zika virus infection.

14. The method of claim 1, wherein the patient sample is whole blood, serum, plasma, saliva, semen, urine, or vaginal fluids.

15. A method for rapid assay of a human patient sample to determine Zika virus infection status in the patient, comprising:
comparing a test signal produced at a first test line on a first lateral flow test strip exposed to the patient sample, a test signal produced at a second test line on a second lateral flow test strip exposed to the patient sample, and a test signal produced at a third test line on the second lateral flow test strip exposed to the patient sample, wherein:
a. the presence of the test signal produced at the first test line indicates the presence of an anti-Zika antibody in the patient sample;
b. the presence of the test signal produced at the second test line indicates the presence of an anti-Zika-IgG in the patient sample; and
c. the presence of the test signal produced at the third test line indicates the presence of an anti-Zika IgM in the patient sample;
wherein the test signal at each of the first, second, and third test lines is produced in five to twenty minutes following application of the patient sample to a sample loading pad on the corresponding lateral flow test strip, and wherein the Zika virus infection status determined is selected from: no Zika virus infection, early Zika virus infection, intermediate Zika virus infection, and late Zika virus infection.

16. A device comprising the assay of claim 12.

17. A test kit comprising the device of claim 16.

18. The method of claim 3, wherein: the first tracer antigen is Zika NS1; the first immobilized Zika detection antigen on the first test line is Zika NS1; and the second tracer antigen is Zika NS1.

19. The method of claim 4, wherein: the first tracer antigen is Zika NS1; the first immobilized Zika detection antigen on the first test line is Zika NS1; and the second tracer antigen is Zika NS1.

20. The method of claim 4, wherein the determination of no Zika virus infection is based on the absence of an antibody test signal at the first test line.
